(19) United States
(12) Patent Application Publication
(10) Pub. No.: US 2007/0072186 A1
(43) Pub. Date: Mar. 29, 2007

(54) Methods and agents for screening for compounds capable of modulating her2 expression

(76) Inventors: Anuradha Mehta, Piscataway, NJ (US); Christopher Robert Trotta, Somerset, NJ (US)

Correspondence Address: ARNOLD & PORTER LLP;ATTN: IP DOCKETING DEPT., 555 TWELFTH STREET, N.W., WASHINGTON, DC, 20004-1206, US

(21) Appl. No.: 10/579,500

(22) PCT Filed: Nov. 17, 2004
(86) PCT No.: PCT/US04/38496

§ 371 Date: Sep. 15, 2006

Related U.S. Application Data
(60) Provisional application No. 60/520,384, filed on Nov. 17, 2003.

(51) Int. Cl. C12Q 1/68; C07H 21/04; C12P 21/06; C07K 14/82

(52) U.S. Cl. 435/6; 435/69.1; 435/320.1; 435/325; 530/350; 536/23.5

(57) ABSTRACT

The invention relates to the fields of screening assays and compounds and methods for altering protein expression and levels of protein. In particular, the invention includes assays to screen for agents capable of modulating expression of Her2 and agents capable of modulating Her2 expression.

### CROSS-REFERENCE TO RELATED APPLICATIONS

This application claims the benefit of U.S. Application No. 60/520,384, filed Nov. 17, 2003, the disclosure of which is hereby incorporated by reference in its entirety.

### INCORPORATION OF SEQUENCE LISTING

A paper copy of the Sequence Listing and a computer readable form of the sequence listing on diskette, containing the file named “Her2 Seq Lst.txt”, which is 17 KB in size (measured in MS-DOS), and which was recorded on Nov. 17, 2003, are herein incorporated by reference.

### BACKGROUND OF THE INVENTION

Regulation of protein expression is often critical for the treatment of diseases, including cancer and other proliferative diseases. Regulation of protein expression can occur at a number of levels, including transcriptional and translational. One area of research has been directed at modulating protein expression by targeting RNA encoding a protein, or fragment thereof, by using anti-sense technology. Another manner in which protein expression is regulated is through modulating translation efficiency. In eukaryotes, untranslated regions (UTRs) are important for overall regulation of translation. A role in regulating translation for regions of a gene such as the 5′ UTR, regions within the 5′ UTR, and the poly(A) tail have been reported. Untranslated regions can be used to modulate gene expression and to identify compounds that affect translation efficiency of a gene.

Her2 occupies a critical position in the biochemical pathways responsible for transduction of mitogenic signals from a variety of growth factor receptors. Overexpression of Her2 is pro-oncogenic and has been implicated in approximately 30% of the solid tumors of the breast, ovary, prostate (Arai et al. (1997) Prostate vol. 30:195-201; Bendell et al., 2003; Takehana et al., 2002). Status of c-erbB-2 in gastric adenocarcinoma: a comparative study of immunohistochemistry, fluorescence in situ hybridization and enzyme-linked immuno-sorbent assay. Int J Cancer vol. 98:833-837), oesophagus (Lam et al., 1998. C-erbB-2 protein expression in oesophageal squamous epithelium from oesophageal squamous cell carcinomas, with special reference to histological grade of carcinoma and pre-invasive lesions. Eur J Surg. Oncol. vol. 24:431-435), and pancreas (Standop et al., (2002) Virchows Arch vol. 441:385-391). Overexpression of Her2 in a wide variety of human cancers has been associated with poor prognosis, neoplastic transformation and aggressive tumor growth (Tzahar et al., (1998) Biophys Acta vol. 1377:M25-M37). Her2 positive status in stomach and other cancers is directly correlated with the metastatic potential and spread of the disease.

Her2 polypeptide levels within a cell are regulated, at least in part, at the transcriptional and translational level. There are at least two elements within the Her2 5′ UTR that have been reported to be strong regulators of polypeptide levels. A 5′ her2 UTR typically includes a region of GC-rich sequence between residues 65 and 150 of the 5′ her2 UTR. In addition, a 5′ her2 UTR typically includes a short upstream open reading frame (uORF) from residues 153 to 173 of the 5′ her2 UTR.

Therapeutics that decrease Her2 polypeptide levels within a cell would be valuable as drugs for the treatment of conditions such as cancer and other proliferative diseases. Current, anti-Her2 antibodies inhibit cancer growth in only 20-25% of Her2 positive cases and cannot access intracellular pools of Her2. Thus, new, innovative drugs with better efficacy and tolerability, specifically targeting Her2 translation, can help in the design of more effective combination therapy treatments.

### BRIEF SUMMARY OF THE INVENTION

To address this need, the present invention includes and provides agents and methods useful in screening for compound capable of modulating gene expression, as well as hybrid molecules. Unique nucleic acids are disclosed that include, without limitation, a specific and unique nucleic acid sequence of the untranslated region 3′ UTR of the Her2 gene, SEQ ID NO: 1, which has been identified as sufficient and useful to reduce Her2 protein expression in vitro and in vivo.

The present invention provides a method comprising: (a) providing a reporter gene linked to an untranslated region comprising SEQ ID NO: 1 and a compound; and (b) detecting expression of said reporter gene, wherein expression of said reporter gene is altered relative to expression of a reporter gene not linked to an untranslated region comprising SEQ ID NO: 1.

The present invention also provides a method comprising: (a) providing a reporter gene linked to an untranslated region selected from the group consisting of a nucleic acid sequence consisting or comprising SEQ ID NO: 1 and SEQ ID NOs: 7-22 and a compound; and (b) detecting expression of said reporter gene, wherein expression of said reporter gene is altered relative to expression of a reporter gene not linked to an untranslated region comprising an untranslated region selected from the group consisting of a nucleic acid sequence consisting or comprising SEQ ID NO: 1 and SEQ ID NOs: 7-22.

The present invention also provides a method comprising: (a) providing a reporter gene linked to an untranslated region from a target gene and a compound, wherein said untranslated region from a target gene is linked to SEQ ID NO: 1; (b) detecting expression of said linked reporter gene; (c) providing a reporter gene not linked to an untranslated region comprising SEQ ID NO: 1 and a compound; and (d) detecting expression of said not linked reporter gene.

The present invention also provides a method comprising: (a) providing a reporter gene linked to an untranslated region from a target gene and a compound, wherein said untranslated region from a target gene is linked to an untranslated region selected from the group consisting of a nucleic acid sequence consisting or comprising SEQ ID NO: 1 and SEQ ID NOs: 7-22; (b) detecting expression of said linked reporter gene; (c) providing a reporter gene not linked to an untranslated region selected from the group consisting of a nucleic acid sequence consisting or comprising SEQ ID NO: 1 and SEQ ID NOs: 7-22 and a compound; and (d) detecting expression of said not linked reporter gene.

The present invention also provides a method comprising: (a) providing a reporter gene linked to an untranslated region from a target gene and a compound, wherein said untranslated region from a target gene is linked to an untranslated region selected from the group consisting of a nucleic acid sequence consisting or comprising SEQ ID NO: 1 and SEQ ID NOs: 7-22; and (b) detecting expression of said reporter gene, wherein said expression of said reporter gene is greater relative to expression of a reporter gene not linked to an untranslated region selected from the group consisting of a nucleic acid sequence consisting or comprising SEQ ID NO: 1 and SEQ ID NOs: 7-22.

The present invention also provides a method comprising: (a) providing a reporter gene linked to an untranslated region from a target gene and a compound, wherein said untranslated region from a target gene is linked to SEQ ID NO: 1; and (b) detecting expression of said reporter gene, wherein said expression of said reporter gene is greater relative to expression of a reporter gene not linked to SEQ ID NO: 1.

The present invention also provides a method comprising: (a) providing a reporter gene linked to an untranslated region from a target gene and a compound, wherein said untranslated region from a target gene is linked to an untranslated region selected from the group consisting of a nucleic acid sequence consisting or comprising SEQ ID NO: 1 and SEQ ID NOs: 7-22; and (b) detecting expression of said reporter gene, wherein said expression of said reporter gene is greater relative to expression of a reporter gene not linked to an untranslated region selected from the group consisting of a nucleic acid sequence consisting or comprising SEQ ID NO: 1 and SEQ ID NOs: 7-22.

The present invention provides and includes a cell line comprising a reporter gene linked to an untranslated region comprising SEQ ID NO: 1.

The present invention provides and includes a cell line comprising a reporter gene linked to an untranslated region comprising an untranslated region selected from the group consisting of a nucleic acid sequence consisting or comprising SEQ ID NO: 1 and SEQ ID NOs: 7-22.

The present invention provides and includes a hybrid of a compound and a nucleic acid molecule comprising SEQ ID NO: 1, wherein said compound is capable of inhibiting expression of a reporter gene linked to said nucleic acid molecule comprising SEQ ID NO: 1 relative to expression of a reporter gene not linked to a nucleic acid molecule comprising SEQ ID NO: 1.

The present invention provides and includes a hybrid of a compound and a nucleic acid molecule comprising SEQ ID NO: 1, wherein said compound is capable of inhibiting expression of a reporter gene linked to said nucleic acid molecule comprising an untranslated region selected from the group consisting of a nucleic acid sequence consisting or comprising SEQ ID NO: 1 and SEQ ID NOs: 7-22 relative to expression of a reporter gene not linked to a nucleic acid molecule comprising an untranslated region selected from the group consisting of a nucleic acid sequence consisting or comprising SEQ ID NO: 1 and SEQ ID NOs: 7-22.

The present invention provides a hybrid of a compound and a nucleic acid molecule comprising SEQ ID NO: 1, wherein said compound is capable of preferentially binding said nucleic acid molecule relative to a nucleic acid molecule not comprising SEQ ID NO: 1.

The present invention provides a hybrid of a compound and a nucleic acid molecule comprising an untranslated region selected from the group consisting of a nucleic acid sequence consisting or comprising SEQ ID NO: 1 and SEQ ID NOs: 7-22, wherein said compound is capable of preferentially binding said nucleic acid molecule relative to a nucleic acid molecule not comprising an untranslated region selected from the group consisting of a nucleic acid sequence consisting or comprising SEQ ID NO: 1 and SEQ ID NOs: 7-22.

The present invention provides and includes a substantially purified nucleic acid molecule comprising between 95% and 100% sequence identity with a nucleic acid molecule of SEQ ID NO: 1, or a fragment thereof, or a complement of either.

The present invention provides and includes a substantially purified nucleic acid molecule comprising between 95% and 100% sequence identity with a nucleic acid molecule of an untranslated region selected from the group consisting of a nucleic acid sequence consisting or comprising SEQ ID NO: 1 and SEQ ID NOs: 7-22, or a fragment thereof, or a complement of either.

The present invention also provides a method for identifying a compound that modulates reporter gene expression comprising: (a) providing a reporter gene linked to an untranslated region comprising SEQ ID NO: 1 and a cellular extract; and (b) detecting expression of said reporter gene, wherein said compound modulates expression of said reporter gene relative to expression of a reporter gene not linked to an untranslated region comprising SEQ ID NO: 1.

The present invention also provides a method for identifying a compound that modulates reporter gene expression comprising: (a) providing a reporter gene linked to an untranslated region comprising an untranslated region selected from the group consisting of a nucleic acid sequence consisting or comprising SEQ ID NO: 1 and SEQ ID NOs: 7-22 and a cellular extract; and (b) detecting expression of said reporter gene, wherein said compound modulates expression of said reporter gene relative to expression of a reporter gene not linked to an untranslated region comprising an untranslated region selected from the group consisting of a nucleic acid sequence consisting or comprising SEQ ID NO: 1 and SEQ ID NOs: 7-22.

The present invention also provides variants of SEQ ID NO: 1, including fragments of SEQ ID NO: 1 with deletions from the 5′ end, the 3′ end, the 5′ and 3′ ends, and internal deletions. Also provided are point variants, nonsense variants, and sense variants of SEQ ID NO: 1. Such variants can include naturally occurring mutants of a Her 2 3′ UTR. Such variants can be non-naturally occurring variants.

The present invention also provides variants of an untranslated region selected from the group consisting of a nucleic acid sequence consisting or comprising SEQ ID NO: 1 and SEQ ID NOs: 7-22, including fragments of an untranslated region selected from the group consisting of a nucleic acid sequence consisting or comprising SEQ ID NO: 1 and SEQ ID NOs: 7-22 with deletions from the 5′ end, the 3′ end, the 5′ and 3′ ends, and internal deletions. Also provided are point variants, nonsense variants, and sense variants of an untranslated region selected from the group consisting of a nucleic acid sequence consisting or comprising SEQ ID NO: 1 and SEQ ID NOs: 7-22. Such variants can include naturally occurring mutants of a Her 2 3′ UTR. Such variants can be non-naturally occurring variants.

The present invention provides and includes a cell line expressing a reporter gene linked to an untranslated region comprising SEQ ID NO: 1 and an untranslated region from a target gene selected from the group consisting of HIF-1α, Vascular Endothelial Growth Factor (VEGF), X-linked inhibitor of apoptosis (XIAP), Survivin, PTP1b, EGFR, TNF-α, Mdm-2, Ship-2, and G-CSF.

The present invention provides and includes a cell line expressing a reporter gene linked to an untranslated region comprising an untranslated region selected from the group consisting of a nucleic acid sequence consisting or comprising SEQ ID NO: 1 and SEQ ID NOs: 7-22 and an untranslated region from a target gene selected from the group consisting of HIF-1α, Vascular Endothelial Growth Factor (VEGF), X-linked inhibitor of apoptosis (XIAP), Survivin, PTP1b, EGFR, TNF-α, Mdm-2, Ship-2, and G-CSF.

The present invention provides and includes a cell line expressing a target gene selected from the group consisting of HIF-1α, Vascular Endothelial Growth Factor (VEGF), X-linked inhibitor of apoptosis (XIAP), Survivin, PTP1b, EGFR, TNF-α, Mdm-2, Ship-2, and G-CSF linked to an untranslated region comprising SEQ ID NO: 1.

The present invention provides and includes a cell line expressing a target gene selected from the group consisting of HIF-1α, Vascular Endothelial Growth Factor (VEGF), X-linked inhibitor of apoptosis (XIAP), Survivin, PTP1b, EGFR, TNF-α, Mdm-2, Ship-2, and G-CSF linked to an untranslated region comprising an untranslated region selected from the group consisting of a nucleic acid sequence consisting or comprising SEQ ID NO: 1 and SEQ ID NOs: 7-22.

The present invention also provides a method for reducing protein levels of Her2 in a cell by addition of a nucleic acid comprising SEQ ID NO: 1, complement thereof, or fragment of either, to the cell.

The present invention also provides a method for reducing protein levels of Her2 in a cell by addition of a nucleic acid comprising an untranslated region selected from the group consisting of a nucleic acid sequence consisting or comprising SEQ ID NO: 1 and SEQ ID NOs: 7-22, complement thereof, or fragment of either, to the cell.

The present invention provides a cell line comprising a UTR comprising SEQ ID NO: 1 linked to a heterologous sequence.

The present invention provides a cell line comprising a UTR comprising an untranslated region selected from the group consisting of a nucleic acid sequence consisting or comprising SEQ ID NO: 1 and SEQ ID NOs: 7-22 linked to a heterologous sequence.

The present invention also provides a method of modulating protein expression levels of a gene comprising: (a) providing a compound; and (b) altering the structure of an RNA molecule comprising a gene and SEQ ID NO: 1.

The present invention also provides a method of modulating protein expression levels of a gene comprising: (a) providing a compound; and (b) altering the structure of an RNA molecule comprising a gene and an untranslated region selected from the group consisting of a nucleic acid sequence consisting or comprising SEQ ID NO: 1 and SEQ ID NOs: 7-22.

The present invention also provides a compound that alters the structure of an RNA molecule, wherein said RNA molecule comprises a gene linked to SEQ ID NO: 1, whereby protein expression levels of said reporter gene are decreased relative to a RNA molecule comprises a gene not linked to SEQ ID NO: 1.

The present invention also provides a compound that alters the structure of an RNA molecule, wherein said RNA molecule comprises a gene linked to an untranslated region selected from the group consisting of a nucleic acid sequence consisting or comprising SEQ ID NO: 1 and SEQ ID NOs: 7-22, whereby protein expression levels of said reporter gene are decreased relative to a RNA molecule comprises a gene not linked to an untranslated region selected from the group consisting of a nucleic acid sequence consisting or comprising SEQ ID NO: 1 and SEQ ID NOs: 7-22.

The present invention also provides a screen for compounds not previously known to alter Her2 protein levels.

### BRIEF DESCRIPTION OF THE DRAWINGS

FIG. 1 shows inhibition of translation for a capped-5′+3′ UTR of Her 2 linked to a luciferase gene in the presence of a 5-fold molar excess of a 73-residue region from a 3′ UTR of Her2 (SEQ ID NO: 1).

FIG. 2 shows schematics of some constructs used in Table 2.

FIG. 3 shows translational regulation of luciferase protein levels as a function of linkage to UTRs from Her2 and cellular background.

FIG. 4 shows schematics of some constructs used in FIG. 3.

FIG. 5 shows schematics of some illustrative TRE constructs.

FIG. 6 shows schematics of some illustrative 3′ her2 UTR variant constructs.

FIG. 7 shows the activity of Her2 3′ UTRs in chimeric contructs that contain a Ship 5′ UTR (10 amino acid uORF).

FIG. 8 shows the translation of a 5′ Her2-Luciferase-3′ Her2 construct.

FIG. 9A shows the translation of a capped 5′ Her2-Luciferase-3′ Her2 RNA in the presence of competitor RNA.

FIG. 9B shows the translation of a capped vector-only, control RNA in the presence of competitor RNA.

FIG. 10A shows a 48-kDa polypeptide, which is capable of crosslinking to constructs containing TRE1.

FIG. 10B shows competition with nucleic acid molecules that are capable of preventing binding of the 48-kDa polypeptide to the Her2 3′ UTR.

FIG. 10C shows that the relative abundance of the 48-kDa polypeptide correlates with Her2 expression.

### DESCRIPTION OF THE NUCLEIC ACID SEQUENCES

SEQ ID NO: 1 sets forth a nucleic acid sequence of a TRE1.

SEQ ID NO: 2 sets forth a nucleic acid sequence of a naturally occurring Her2 open reading frame.

SEQ ID NO: 3 sets forth a nucleic acid sequence of a naturally occurring Her2 3′ UTR.

SEQ ID NO: 4 sets forth a nucleic acid sequence of a naturally occurring Her2 3′ UTR.

SEQ ID NO: 5 sets forth a nucleic acid sequence of a Her2 3′ UTR variant of 310-residues.

SEQ ID NO: 6 sets forth a nucleic acid sequence of a naturally occurring Her2 5′ UTR

SEQ ID NO: 7 sets forth a nucleic acid sequence of a TRE17.

SEQ ID NO: 8 sets forth a nucleic acid sequence of a TRE2.

SEQ ID NO: 9 sets forth a nucleic acid sequence of a TRE3.

SEQ ID NO: 10 sets forth a nucleic acid sequence of a TRE4.

SEQ ID NO: 11 sets forth a nucleic acid sequence of a TRE5.

SEQ ID NO: 12 sets forth a nucleic acid sequence of a TRE6.

SEQ ID NO: 13 sets forth a nucleic acid sequence of a TRE7.

SEQ ID NO: 14 sets forth a nucleic acid sequence of a TRE8.

SEQ ID NO: 15 sets forth a nucleic acid sequence of a TRE9.

SEQ ID NO: 16 sets forth a nucleic acid sequence of a TRE10.

SEQ ID NO: 17 sets forth a nucleic acid sequence of a TRE11.

SEQ ID NO: 18 sets forth a nucleic acid sequence of a TRE12.

SEQ ID NO: 19 sets forth a nucleic acid sequence of a TRE13.

SEQ ID NO: 20 sets forth a nucleic acid sequence of a TRE14.

SEQ ID NO: 21 sets forth a nucleic acid sequence of a TRE15.

SEQ ID NO: 22 sets forth a nucleic acid sequence of a TRE16.

SEQ ID NO: 23 sets forth a nucleic acid sequence of a Her2 3′ UTR variant.

SEQ ID NO: 24 sets forth a nucleic acid sequence of a Her2 3′ UTR variant.

SEQ ID NO: 25 sets forth a nucleic acid sequence of a Her2 3′ UTR variant.

SEQ ID NO: 26 sets forth a nucleic acid sequence of a Her2 3′ UTR variant.

SEQ ID NO: 27 sets forth a nucleic acid sequence of a Her2 3′ UTR variant.

SEQ ID NO: 28 sets forth a nucleic acid sequence of a Her2 3′ UTR variant.

SEQ ID NO: 29 sets forth a nucleic acid sequence of a Her2 3′ UTR variant with nucleotides 1-110 deleted at the 5′ end.

SEQ ID NO: 30 sets forth a nucleic acid sequence of a Her2 3′ UTR variant.

Definitions

As used herein, the term “construct” refers to a nucleic acid molecule having an untranslated region, a coding sequence, or both inserted into a vector.

As used herein, the term “derivative” refers to a chemical substance related structurally to another substance and can, at least theoretically, be formed from another substance.

As used herein, the term “hybrid” is a hybrid formed between two non-identical molecules that are non-covalently attached.

As used herein, the term “mammalian cancer cell” refers to a cell derived from a mammal that does not respond appropriately to external cues.

As used herein, the term “poly(A) tail” refers to a polyadenylic acid tail that is added to the 3′ end of a pre-mRNA.

As used herein, a “reporter gene” is any gene whose expression can be measured, except a naturally occurring Her2 gene located upstream from SEQ ID NO: 1. An example of a naturally occurring Her2 gene is exemplified in SEQ ID NO: 2. In a preferred embodiment, a reporter gene can have a previously determined reference range of detectable expression.

As used herein, the term “RNA induced gene silencing, or RNA interference (RNAi)” refers to the mechanism of double-stranded RNA (dsRNA) introduced into a system to silence protein expression.

As used herein, the term “specifically bind” means that a compound binds to another compound in a manner different from a similar type of compounds, e.g. in terms of affinity, avidity, and the like. In a non-limiting example, more binding occurs in the presence of a competing reagent, such as casein. In another non-limiting example, antibodies that specifically bind a target protein should provide a detection signal at least 2-, 5-, 10-, or 20-fold higher relative to a detection signal provided with other molecules when used in Western blots or other immunochemical assays. In an alternative non-limiting example, a nucleic acid can specifically bind its complementary nucleic acid molecule. In another non-limiting example, a transcription factor can specifically bind a particular nucleic acid sequence.

As used herein, the term “secondary structure” means the alpha-helical, beta-sheet, random coil, beta turn structures and helical nucleic acid structures that occur in proteins, peptide nucleic acids, compounds comprising modified nucleic acids, compounds comprising modified amino acids and other types of compounds as a result of, at least, the compound's composition.

As used herein, the term “small-molecule” and analogous terms include, but are not limited to organic or inorganic compounds (i.e., including heteroorganic and organometallic compounds).

As used herein, a “translational regulatory element (TRE)” is not SEQ ID NO: 2, but may be a fragment thereof.

As used herein, the term “UTR” refers to the untranslated region of a gene.

As used herein, the term “vector” refers to a nucleic acid molecule functioning as the backbone of a construct.

### DETAILED DESCRIPTION OF THE INVENTION

The present invention includes and utilizes the fact that an untranslated region (UTR) is capable of modulating expression of a gene and that such modulation of expression is capable of being altered or modulated by the addition of compounds. In a preferred embodiment, a UTR is a region of a mRNA that is not translated into protein. In a more preferred embodiment, the UTR is a 5′ UTR, i.e., upstream of the coding region, or a 3′ UTR, i.e., downstream of the coding region. In another embodiment, the term UTR corresponds to a reading frame within the mRNA that is not translated.

Moreover, the present invention includes and provides agents and methods useful in screening for compound capable of modulating gene expression and also hybrid molecules. Unique nucleic acids disclosed herein include, without limitation, a specific and unique nucleic acid sequence of the untranslated region 3′ UTR of the Her2 gene, SEQ ID NO: 1, which have been identified as sufficient and useful to reduce Her2 protein expression in vitro and in vivo.

Agents

The terms “isolated” or “substantially purified” refer to material that is substantially or essentially free from components which normally accompany it as found in its native state. Purity and homogeneity are typically determined using analytical chemistry techniques such as polyacrylamide gel electrophoresis or high performance liquid chromatography. A protein that is the predominant species present in a preparation is substantially purified. The term isolated or substantially purified denotes that a nucleic acid or protein gives rise to essentially one band in an electrophoretic gel. Particularly, it means that the nucleic acid or protein is at least 85% pure, optionally at least 95% pure, and optionally at least 99% pure.

Nucleic Acid Agents and Constructs

One skilled in the art may refer to general reference texts for detailed descriptions of known techniques discussed herein or equivalent techniques. These texts include Ausubel et al., Current Protocols in Molecular Biology, John Wiley and Sons, Inc. (1995); Sambrook et al., Molecular Cloning, A Laboratory Manual (2d ed.), Cold Spring Harbor Press, Cold Spring Harbor, N.Y. (1989); Birren et al., Genome Analysis: A Laboratory Manual, volumes 1 through 4, Cold Spring Harbor Press, Cold Spring Harbor, N.Y. (1997-1999). These texts can, of course, also be referred to in making or using an aspect of the invention.

3′ Her2 UTRs

The present invention includes nucleic acid molecules that comprise or consist of a translational regulatory element (TRE) including SEQ ID NO: 1, variants of SEQ ID NO: 1, and fragments and complements of all.

A TRE of the present invention can differ from any of the residues in SEQ ID NO: 1 in that the nucleic acid sequence has been deleted, substituted, or added in a manner that does not alter the function. In another aspect of the present invention, a TRE of the present invention consists or comprises SEQ ID NO: 7, variants of SEQ ID NO: 7, and fragments and complements of all. In another aspect of the present invention, a TRE of the present invention consists or comprises SEQ ID NOs: 8-22, variants of SEQ ID NOs: 8-22, and fragments and complements of all.

A TRE of the present invention can differ from any of the residues in an untranslated region selected from the group consisting of a nucleic acid sequence consisting or comprising SEQ ID NO: 1 and SEQ ID NOs: 7-22 in that the nucleic acid sequence has been deleted, substituted, or added in a manner that does not alter the function.

In another aspect of the present invention, a Her2 3′ UTR of the present invention consists or comprises SEQ ID NOs: 23-28 and SEQ ID NO: 29, variants of SEQ ID NOs: 23-28 and SEQ ID NO: 29, and fragments and complements of all.

The present invention provides nucleic acid molecules that hybridize to the above-described nucleic acid molecules. In a preferred aspect, the nucleic acid molecule hybridizes to a nucleic acid molecule selected from the group consisting of a nucleic acid sequence consisting or comprising SEQ ID NO: 1, SEQ ID NOs: 7-22, and complements thereof. Nucleic acid hybridization is a technique well known to those of skill in the art of DNA manipulation. The hybridization properties of a nucleic acid molecule are an indication of their similarity or identity. The nucleic acid molecules preferably hybridize, under moderate or high stringency conditions, with a nucleic acid sequence selected from SEQ ID NO: 1 and complements thereof. Fragments of these sequences are also contemplated.

In another aspect, the nucleic acid molecules preferably hybridize, under moderate or high stringency conditions, with a nucleic acid sequence selected from the group consisting of SEQ ID NO: 1 and its complement.

The hybridization conditions typically involve nucleic acid hybridization in about 0.1× to about 10×SSC (diluted from a 20×SSC stock solution containing 3 M sodium chloride and 0.3 M sodium citrate, pH 7.0 in distilled water), about 2.5× to about 5× Denhardt's solution (diluted from a 50× stock solution containing 1% (w/v) bovine serum albumin, 1% (w/v) Ficoll® (Amersham Biosciences Inc., Piscataway, N.J.), and 1% (w/v) polyvinylpyrrolidone in distilled water), about 10 mg/ml to about 100 mg/ml salmon sperm DNA, and about 0.02% (w/v) to about 0.1% (w/v) SDS, with an incubation at about 20° C. to about 70° C. for several hours to overnight.

In a preferred aspect, the moderate stringency hybridization conditions are provided by 6×SSC, 5× Denhardt's solution, 100 mg/ml salmon sperm DNA, and 0.1% (w/v) SDS, with an incubation at 55° C. for several hours. The moderate stringency wash conditions are about 0.02% (w/v) SDS, with an incubation at about 55° C. overnight. In a more preferred aspect, the high stringency hybridization conditions are about 2×SSC, about 3× Denhardt's solution, and about 10 mg/ml salmon sperm DNA. The high stringency wash conditions are about 0.05% (w/v) SDS, with an incubation at about 65° C. overnight.

In an embodiment, the nucleic acid molecule comprises a nucleic acid sequence that is greater than 85% identical, and more preferably greater than 86, 87, 88, 89, 90, 91, 92, 93, 94, 95, 96, 97, 98, or 99% identical to a nucleic acid sequence selected from the group consisting of SEQ ID NO: 1 and SEQ ID NOs: 7-22, complements thereof, and fragments of any of these sequences.

The percent identity is preferably determined using the “Best Fit” or “Gap” program of the Sequence Analysis Software Package™ (Version 10; Genetics Computer Group, Inc., University of Wisconsin Biotechnology Center, Madison, Wis.). “Gap” utilizes the algorithm of Needleman and Wunsch to find the alignment of two sequences that maximizes the number of matches and minimizes the number of gaps. “BestFit” performs an optimal alignment of the best segment of similarity between two sequences and inserts gaps to maximize the number of matches using the local homology algorithm of Smith and Waterman. The percent identity calculations may also be performed using the Megalign program of the LASERGENE bioinformatics computing suite (default parameters, DNASTAR Inc., Madison, Wis.). The percent identity is most preferably determined using the “Best Fit” program using default parameters.

Fragment nucleic acid molecules can contain significant portions of, or indeed most of, SEQ ID NO: 1. In an embodiment, the fragments are between 73 and 60 consecutive residues, 75 and 50 consecutive residues, 50 and 25 consecutive residues, or 20 and 10 consecutive residues long of a nucleic molecule of the present invention. In another embodiment, the fragment comprises at least 20, 30, 40, 50, 60, or 70 consecutive residues of SEQ ID NO: 1. In a particularly preferred embodiment, a fragment nucleic acid molecule is capable of selectively hybridizing to SEQ ID NO: 1.

Any of a variety of methods may be used to obtain one or more of the above-described nucleic acid molecules. Automated nucleic acid synthesizers may be employed for this purpose. In lieu of such synthesis, the disclosed nucleic acid molecules may be used to define a pair of primers that can be used with the polymerase chain reaction (PCR) to amplify and obtain any desired nucleic acid molecule or fragment.

In one aspect of the present invention, a TRE comprises at least one of the first, second, third, forth, fifth or sixth most 3′ residues of SEQ ID NO: 1. In another aspect of the present invention, a TRE comprises or consists of SEQ ID NO: 1 and SEQ ID NOs: 7-22, and fragments and complements of all.

Short nucleic acid sequences having the ability to specifically hybridize to complementary nucleic acid sequences may be produced and utilized in the present invention, e.g., as probes to identify the presence of a complementary nucleic acid sequence in a given sample. Alternatively, the short nucleic acid sequences may be used as oligonucleotide primers to amplify or mutate a complementary nucleic acid sequence using PCR technology. These primers may also facilitate the amplification of related complementary nucleic acid sequences (e.g., related sequences from other species).

Use of these probes or primers may greatly facilitate the identification of transgenic cells or organisms that contain the presently disclosed structural nucleic acid sequences. Such probes or primers may also, for example, be used to screen cDNA, mRNA, or genomic libraries for additional nucleic acid sequences related to or sharing homology with the presently disclosed promoters and structural nucleic acid sequences. The probes may also be PCR probes, which are nucleic acid molecules capable of initiating a polymerase activity while in a double-stranded structure with another nucleic acid.

A primer or probe is generally complementary to a portion of a nucleic acid sequence that is to be identified, amplified, or mutated and of sufficient length to form a stable and sequence-specific duplex molecule with its complement. The primer or probe preferably is about 10 to about 200 residues long, more preferably is about 10 to about 100 residues long, even more preferably is about 10 to about 50 residues long, and most preferably is about 14 to about 30 residues long.

The primer or probe may, for example without limitation, be prepared by direct chemical synthesis, by PCR (U.S. Pat. Nos. 4,683,195 and 4,683,202), or by excising the nucleic acid specific fragment from a larger nucleic acid molecule. Various methods for determining the sequence of PCR probes and PCR techniques exist in the art. Computer-generated searches using programs such as Primer3 (www-genome.wi.mit.edu/cgi-bin/primer/primer3.cgi), STSPipeline (www-genome.wi.mit.edu/cgi-bin/www-STS_Pipeline), or GeneUp (Pesole et al., BioTechniques 25:112-123, 1998), for example, can be used to identify potential PCR primers.

Furthermore, sequence comparisons can be done to find nucleic acid molecules of the present invention based on secondary structure homology. Several methods and programs are available to predict and compare secondary structures of nucleic acid molecules, for example, GeneBee (available on the world wide web at genebee.msu.su/services/rna2_reduced.html); the Vienna RNA Package (available on the world wide web at tbi.univie.ac.at/˜ivo/RNA/); SstructView (available on the world wide web at the Stanford Medical Informatics website, under: projects/helix/sstructview/home.html and described in “RNA Secondary Structure as a Reusable Interface to Biological Information Resources.” 1997. Gene vol. 190GC59-70). For example, comparisons of secondary structure are preformed in Le et al., A common RNA structural motif involved in the internal initiation of translation of cellular mRNAs. 1997. Nuc. Acid. Res. vol. 25(2):362-369, the disclosure of which is hereby incorporated by reference.

Constructs of the Present Invention

The present invention includes without limitation and provides nucleic acid constructs. It is understood that any of the constructs and other nucleic acid agents of the present invention can be either DNA or RNA. Moreover, any of the nucleic acid molecules or constructs of the present invention can be used in combination with a method of the present invention.

Vectors

Exogenous genetic material may be transferred into a host cell by use of a vector or construct designed for such a purpose. Any of the nucleic acid sequences of the present invention may be introduced into a recombinant vector to make a construct of the present invention. A vector may be a linear or a closed circular plasmid. A vector system may be a single vector or plasmid or two or more vectors or plasmids that together contain the total DNA to be introduced into the genome of the host. Means for preparing recombinant vectors are well known in the art.

Vectors suitable for replication in mammalian cells may include viral replicons, or sequences that insure integration of the appropriate sequences encoding HCV epitopes into the host genome. For example, another vector used to express foreign DNA is vaccinia virus. Such heterologous DNA is generally inserted into a gene that is non-essential to the virus, for example, the thymidine kinase gene (tk), which also provides a selectable marker. Expression of the HCV polypeptide then occurs in cells or animals that are infected with the live recombinant vaccinia virus.

In general, plasmid vectors containing replicon and control sequences that are derived from species compatible with the host cell are used in connection with bacterial hosts. The vector ordinarily carries a replication site, as well as marking sequences that are capable of providing phenotypic selection in transformed cells. For example, E. coli is typically transformed using a construct with a backbone derived from a vector, such as pBR322, which contains genes for ampicillin and tetracycline resistance and thus provides easy means for identifying transformed cells. The pBR322 plasmid, or other microbial plasmid or phage, also generally contains, or is modified to contain, promoters that can be used by the microbial organism for expression of the selectable marker genes.

In a preferred aspect of the present invention, a vector of the present invention consists or comprises SEQ ID NOs: 23-28 and SEQ ID NO: 29, variants of SEQ ID NOs: 23-28 and SEQ ID NO: 29, and fragments and complements of all.

Promoters

A construct can include a promoter, e.g., a recombinant vector typically comprises, in a 5′ to 3′ orientation: a promoter to direct the transcription of a nucleic acid molecule of interest.

In a preferred aspect of the present invention, a construct can include a mammalian promoter and can be used to express a nucleic acid molecule of choice. As used herein, a “mammalian promoter” refers to a promoter functional in a mammalian cell. A number of promoters that are active in mammalian cells have been described in the literature. A promoter can be selected on the basis of the cell type into which the vector will be inserted.

A preferred promoter of the present invention is a Her2 promoter. In addition to Her2 promoters described previously (for example, Yu et al., 2002. Identification of a minimal c-erbB-2 promoter region that mediates preferential expression of a linked foreign gene in human breast cancer cells. J. Oncol. vol. 20(3):607-610; and Nezu, et al., 1999. Identification of a novel promoter and exons of the c-ERBB-2 gene, Biochem. Biophys. Res. Commun. vol. 258 (3):499-505, the disclosures of which are hereby incorporated by reference), other promoter sequences can be utilized in a construct or other nucleic acid molecule. Suitable promoters include, but are not limited to, those described herein.

Suitable promoters for mammalian cells are known in the art and include viral promoters, such as those from Simian Virus 40 (SV40), Rous sarcoma virus (RSV), adenovirus (ADV), cytomegalovirus (CMV), and bovine papilloma virus (BPV), and the parvovirus B19p6 promoter as well as mammalian cell-derived promoters. A number of viral-based expression systems can be used to express a reporter gene in mammalian host cells. For example, if an adenovirus is used as an expression vector, sequences encoding a reporter gene can be ligated into an adenovirus transcription/translation complex comprising the late promoter and tripartite leader sequence.

Other examples of preferred promoters include tissue-specific promoters and inducible promoters. Other preferred promoters include the hematopoietic stem cell-specific, e.g., CD34, glucose-6-phosphotase, interleukin-1 alpha, CD11c integrin gene, GM-CSF, interleukin-5R alpha, interleukin-2, c-fos, h-ras and DMD gene promoters. Other promoters include the herpes thymidine kinase promoter, and the regulatory sequences of the metallothionein gene.

Inducible promoters suitable for use with bacteria hosts include the β-lactamase and lactose promoter systems, the arabinose promoter system, alkaline phosphatase, a tryptophan (trp) promoter system and hybrid promoters such as the tac promoter. However, other known bacterial inducible promoters are suitable. Promoters for use in bacterial systems also generally contain a Shine-Dalgarno sequence operably linked to the DNA encoding the polypeptide of interest.

A promoter can also be selected on the basis of their regulatory features, e.g., enhancement of transcriptional activity, inducibility, tissue specificity, and developmental stage-specificity. A promoter can work in vitro, for example the T7-promoter. Particularly preferred promoters can also be used to express a nucleic acid molecule of the present invention in a nonhuman mammal. Additional promoters that may be utilized are described, for example, in Bemoist and Chambon, Nature 290:304-310 (1981); Yamamoto et al., Cell 22:787-797 (1980); Wagner et al., PNAS 78:1441-1445 (1981); Brinster et al., Nature 296:39-42 (1982).

Reporter Genes

As used herein, a “reporter gene” is any gene whose expression can be measured, except a naturally occurring Her2 gene located upstream from SEQ ID NO: 1. An example of a naturally occurring Her2 gene is exemplified in SEQ ID NO: 2. In a preferred embodiment, a reporter gene can have a previously determined reference range of detectable expression.

A reporter gene of the present invention encoding a protein, a fragment thereof, or a polypeptide, may also be linked to a propeptide encoding region. A propeptide is an amino acid sequence found at the amino terminus of a proprotein or proenzyme. Cleavage of the propeptide from the proprotein yields a mature biochemically active protein. The resulting polypeptide is known as a propolypeptide or proenzyme (or a zymogen in some cases). Propolypeptides are generally inactive and can be converted to mature active polypeptides by catalytic or autocatalytic cleavage of the propeptide from the propolypeptide or proenzyme.

A reporter gene can express a selectable or screenable marker. Selectable markers may also be used to select for organisms or cells that contain exogenous genetic material. Examples of such include, but are not limited to: a neo gene, which codes for kanamycin resistance and can be selected for using kanamycin, GUS, green fluorescent protein (GFP), neomycin phosphotransferase II (nptII), luciferase (LUX), or an antibiotic resistance coding sequence. Screenable markers can be used to monitor expression. Exemplary screenable markers include: a β-glucuronidase or uidA gene (GUS) which encodes an enzyme for which various chromogenic substrates are known; a β-lactamase gene, a gene which encodes an enzyme for which various chromogenic substrates are known (e.g., PADAC, a chromogenic cephalosporin); a luciferase gene; a tyrosinase gene, which encodes an enzyme capable of oxidizing tyrosine to DOPA and dopaquinone which in turn condenses to melanin; and α-galactosidase, which can turn a chromogenic α-galactose substrate.

Included within the terms “selectable or screenable marker genes” are also genes that encode a secretable marker whose secretion can be detected as a means of identifying or selecting for transformed cells. Examples include markers that encode a secretable antigen that can be identified by antibody interaction, or even secretable enzymes, which can be detected utilizing their inherent properties. Secretable proteins fall into a number of classes, including small, diffusible proteins which are detectable, (e.g., by ELISA), or small active enzymes which are detectable in extracellular solution (e.g., α-amylase, β-lactamase, phosphinothricin transferase). Other possible selectable or screenable marker genes, or both, are apparent to those of skill in the art.

A reporter gene can express a fusion protein. As such, said fusion protein can be a fusion of any reporter gene operably linked to another gene, or fragment thereof. For instance, the expressed fusion protein can provide a “tagged” epitope to facilitate detection of the fusion protein, such as GST, GFP, FLAG, or polyHIS. Such fusions preferably encode between 1 and 50 amino acids, more preferably between 5 and 30 additional amino acids, and even more preferably between 5 and 20 amino acids. In one embodiment, a fusion protein can be a fusion protein that includes in whole or in part of a Her2 protein sequence.

Alternatively, the fusion can provide regulatory, enzymatic, cell signaling, or intercellular transport functions. For example, a sequence encoding a signal peptide can be added to direct a fusion protein to a particular organelle within a eukaryotic cell. Such fusion partners preferably encode between 1 and 1000 additional amino acids, more preferably between 5 and 500 additional amino acids, and even more preferably between 10 and 250 amino acids.

Reporter genes can be expressed in vitro or in vivo. In vivo expression can be in a suitable bacterial or eukaryotic host. Suitable methods for expression are described by Sambrook et al., Molecular Cloning: A Laboratory Manual, Second Edition, Cold Spring Harbor Laboratory Press, Cold Spring Harbor, N.Y. (1989); Haymes et al., Nucleic Acid Hybridization, A Practical Approach, IRL Press, Washington, D.C. (1985); or similar texts. Fusion protein or peptide molecules of the invention are preferably produced via recombinant means. These proteins and peptide molecules can be derivatized to contain carbohydrate or other moieties (such as keyhole limpet hemocyanin, etc.).

Linked

As used herein, linked means physically linked, operably linked, or physically and operably linked. As used herein, physically linked means that the physically linked nucleic acid sequences are located on the same nucleic acid molecule, for example a promoter can be physically linked to a reporter gene as part of a construct. In a preferred aspect, the promoter is operably linked to a nucleic acid molecule of the present invention.

UTRs

Agents and constructs of the invention include nucleic acid molecules with an untranslated region (UTR). In a preferred aspect, a UTR refers to a UTR of an mRNA, i.e. the region of the mRNA that is not translated into protein. In a preferred embodiment, a UTR contains one or more regulatory elements that modulate untranslated region-dependent regulation of gene expression. In a particularly preferred embodiment, untranslated region-dependent regulation is mediated by a uORF. In a particularly preferred embodiment, a UTR is a 5′ UTR, i.e., upstream of the coding region, or a 3′ UTR, i.e., downstream of the coding region. In another particularly preferred embodiment, the 5′ UTR includes a Her2 promoter. In a more preferred embodiment, the 5′ UTR includes a Her2 promoter and an upstream open reading frame (uORF). In a preferred aspect, a uORF can code for about 3-25 amino acids. In a preferred embodiment, a uORF codes for about 10 amino acids. An upstream open reading frame (uORF) is upstream of the main open reading frame (main ORF). As used herein, a “main ORF” is any gene with an open reading frame that can be translated. In a preferred embodiment, one or more uORFs are located between about 5 to about 100 residues from the ATG of the main ORF, or between about 10 to about 50 residues from the ATG of the main ORF, or between about 5 to about 25 residues residues from the ATG of the main ORF.

Examples of a main ORF include a reporter gene, a target gene, and a control gene. As used herein, a “target gene” is any gene. In a preferred embodiment, a target gene is a gene operatively linked downstream of a 5′ UTR containing one or more uORFs. As used herein, a “control gene” is any gene. In a preferred embodiment, a control gene is a gene operatively linked downstream of a 5′ UTR that does not contain a uORF.

A UTR of the present invention can be operatively, physically, or operatively and physically linked to a reporter gene. In a preferred embodiment of the present invention, a UTR of the present invention is physically linked to a reporter gene. The physical, operable, or physical and operable linkage may be upstream, downstream, or internal to the reporter gene. As used herein, operably linked means that the operably linked nucleic acid sequences exhibit their deserved function. For example, a promoter can be operably linked to a reporter gene.

In a preferred aspect of the present invention, a UTR of the present invention is a 3′Her2 UTR physically linked downstream of a reporter gene. In a particularly preferred embodiment, Her2 3′ UTR contains or consists of SEQ ID NO: 1 and is physically linked downstream of a reporter gene.

In a preferred aspect of the present invention, a UTR of the present invention is a 5′ her2 UTR physically linked upstream to a reporter gene. In a particularly preferred embodiment, Her2 5′ UTR contains or consists of an upstream open reading frame (uORF) and is physically and operatively linked upstream of a reporter gene. In a more particularly preferred embodiment, Her2 5′ UTR contains or consists of an upstream open reading frame (uORF) and SEQ ID NO: 7 and is physically and operatively linked upstream of a reporter gene.

In a preferred embodiment of the present invention, a UTR of the present invention is physically linked upstream to a reporter gene and another UTR of the present invention is physically linked downstream of the reporter gene. In a particularly preferred embodiment, a UTR of the present invention contains or consists of an upstream open reading frame (uORF) and is physically and operatively linked upstream of a reporter gene and a UTR of the present invention contains or consists of SEQ ID NO: 1 and is physically and operatively linked downstream of a reporter gene.

In a preferred embodiment of the present invention, a UTR of the present invention is physically linked internal to a reporter gene. In a more preferred embodiment of the present invention, a UTR of the present invention containing SEQ ID NO: 1 is physically linked internal to a reporter gene.

In a preferred embodiment of the present invention, a UTR of the present invention is physically linked upstream of a reporter gene and a UTR is physically linked internal to a reporter gene.

In a preferred embodiment of the present invention, a UTR of the present invention is physically linked upstream of a reporter gene and a UTR is physically linked downstream of the reporter gene. In a more preferred embodiment of the present invention, a Her2 5′ UTR of the present invention containing a SEQ ID NO: 1 is physically linked upstream of a reporter gene and a Her2 3′ UTR is physically linked downstream of the reporter gene. Illustrative constructs are set forth in FIGS. 3, 5, and 6.

TREs

While the present invention is directed, in part, to Her2 3′ UTRs, TREs of the present invention can be located in any position within a construct and not limited to the 3′ UTR region of a construct. A TRE of the present invention can be operatively, physically, or operatively and physically linked to a UTR. In an alternative embodiment of the present invention, a TRE of the present invention is a UTR of the present invention.

In a preferred embodiment, a TRE of the present invention is located between about 1000 to about 500 residues upstream from the 5′ end of a mRNA poly(A) tail or polyadenylation signal, between about 500 to about 100 residues upstream from the 5′ end of a mRNA poly(A) tail or polyadenylation signal, or between about 100 to about 60 residues upstream from the 5′ end of a mRNA poly(A) tail or polyadenylation signal. In a most preferred embodiment, the TRE is about 80 residues upstream from the 5′ end of a mRNA poly(A) tail or polyadenylation signal.

In another embodiment, a TRE of the present invention is between about 1000 to about 500 residues downstream from the 3′ end of a main ORF, between about 500 to about 100 residues downstream from the 3′ end of a main ORF, or between about 100 to about 60 residues downstream from the 3′ end of a main ORF. In another embodiment, a UTR is within about 1000 residues upstream from the 5′ end of a main ORF, about 500 residues upstream from the 5′ end of a main ORF, or within about 200 residues upstream from the 5′ end of a main ORF, or about 100 residues upstream from the 5′ end of a main ORF. In another embodiment, a UTR is within the main ORF and between about 1000 to about 500 residues upstream from the 3′ end of a main ORF, between about 500 to about 100 residues upstream from the 3′ end of a main ORF, or between about 100 to about 60 residues upstream from the 3′ end of a main ORF. In a most preferred embodiment, the untranslated region is within 30 residues upstream from the 3′ end of a main ORF.

In another embodiment, a TRE of the present invention is between about 20 to about 5 kilo basepairs downstream from the 5′ start of a main ORF, or between about 10 to about 2 kilo basepairs downstream from the 5′ start of a main ORF, or between about 5 to about 2 kilo basepairs downstream from the 5′ start of a main ORF.

A TRE of the present invention can be linked to a second nucleic acid sequence. In a preferred embodiment, the link can be an operative, physical, or operative and physical linkage to a second nucleic acid sequence. In a preferred embodiment, the second nucleic acid sequence is a UTR. In a more preferred embodiment, the UTR contains an upstream ORF (uORF). A TRE of the present invention can require or may not require an operative, physical, or operative and physical linkage to a second nucleic acid sequence.

In one embodiment of the present invention, an effect of additions, substitutions, deletions of a TRE are only observed in the presence of a linked second nucleic acid sequence. In another embodiment, the effect is an increase in expression or a decrease in protein expression level. In a preferred embodiment, the TRE acts synergistically with the second nucleic acid sequence, which is be operatively, physically, or operatively and physically linked. Linkage of the second nucleic acid sequence and a TRE can increase or decrease expression. the comprising a translational uORF. In a most preferred method, the second nucleic acid sequence is the uORF in the 5′ her2 UTR and the TRE is TRE1. In this embodiment, there is a synergistic increase relative to protein expression with the presence of the her2 uORF in protein expression that occurs when the her2 uORF and the her2 TRE are linked.

Constructs of the present invention can have more or fewer components than described above. For example, constructs of the present invention can include genetic elements, including but not limited to, 3′ transcriptional terminators, 3′ polyadenylation signals, other untranslated nucleic acid sequences, transit or targeting sequences, selectable or screenable markers, promoters, enhancers, and operators, as desired. Constructs of the present invention can also contain a promoterless gene that may utilize an endogenous promoter upon insertion into a host cell chromosome.

Alternatively, sequences encoding nucleic acid molecules of the present invention can be cloned into a vector for the production of an mRNA probe. Such vectors are known in the art, are commercially available, and can be used to synthesize RNA probes in vitro by addition of labeled nucleotides and an appropriate RNA polymerase such as T7, T3, or SP6. These procedures can be conducted using a variety of commercially available kits (for example, Amersham Biosciences Inc., Piscataway, N.J.; and Promega Co, Madison, Wis.).

Modulation of Gene Expression by Nucleic Acid Molecules of the Present Invention

Modulation of gene expression can result in more or less gene expression. In a preferred embodiment, the primary mode of modulated translation of a reporter gene in the presence of constructs of the present invention is not transcript stabilization. Also preferred, modulation in the presence of a construct of the present invention is not due to variation in the 3′ end formation of the processed transcripts. Constructs of the present invention form proper 3′ ends.

In another preferred embodiment, modulation of gene expression can be the result of locating a TRE at an unnatural, physical location within a nucleic acid molecule of the present invention. In an alternate embodiment, modulation of gene expression can be the result of removing about 100-residues, for example without limitation, 100 nucleotides from the 5′ end of a her2 3′ UTR or SEQ ID NO: 29, from within a nucleic acid molecule of the present invention. In another embodiment, the lack of suppression results from a combination of these factors.

In another preferred embodiment, modulation of gene expression can be the result of facilitating translation in the presence of a translational repressor. Expression can be suppressed by translational repressors, such as a very long 5′ UTR, a uORF, one or more small molecules, or by altering the ability of a ribosome to scan a 5′ UTR for the main coding region. A molecule of the present invention can be capable of modulating gene expression preferentially in genes that have cap-dependent translation, poly (A) tails, or have cap-dependent translation and poly (A) tails. In an embodiment of the present invention, modulation of expression is dependent on the presence of a poly(A) tail and a cap, and expression of genes with an IRES is not modulated. In addition, a molecule of the present invention can be capable of modulating gene expression preferentially in cells that over-express the gene of interest. For example, a molecule expressing SEQ ID NO: 1 in a Her-2 over-expressing cell, such as BT474 cell line or a cancer cell, may preferentially modulate protein expression. The methods and compositions of the present invention are not limited by any particular theory.

Many approaches for modulating gene expression using nucleic acid molecules of the present invention are known to one skilled in the art. For example, over-expression of a gene product can be the result from transfection of a construct of the present invention into a mammalian cell. Similarly, down-regulation can be the result from transfection of a construct of the present invention into a mammalian cell. Other non-limiting examples include antisense techniques like RNA interference (RNAi), transgenic animals, hybrids, and ribozymes.

Hybrids

In one aspect of the present invention, a hybrid of a compound and a TRE of the present invention is a hybrid formed between two non-identical molecules. In a preferred aspect, a hybrid can be formed between two nucleic acid molecules. For example, a hybrid can be formed between two ribonucleic acid molecules, between a ribonucleic acid molecule and a deoxyribonucleic acid molecule, or between derivatives of either. In alternative embodiment, a hybrid can be formed between a nucleic acid of the present invention and a non-nucleic acid molecule. In a preferred embodiment, a hybrid can be formed between a nucleic acid molecule and a non-nucleic acid molecule, for example, a polypeptide or a small-molecule.

Ribozymes

In one aspect of the present invention, the activity or expression of a gene is regulated by designing trans-cleaving catalytic RNAs (ribozymes) specifically directed to a nucleic acid molecule of the present invention, for example, SEQ ID NO: 1 and SEQ ID NOs: 7-22.

Ribozymes are RNA molecules possessing endoribonuclease activity. Ribozymes are specifically designed for a particular target, and the target message must contain a specific nucleotide sequence. They are engineered to cleave any RNA species site-specifically in the background of cellular RNA. The cleavage event renders the mRNA unstable and prevents protein expression. Importantly, ribozymes can be used to inhibit expression of a gene of unknown function for the purpose of determining its function in an in vitro or in vivo context, by detecting a phenotypic effect.

One commonly used ribozyme motif is the hammerhead, for which the substrate sequence requirements are minimal. Design of the hammerhead ribozyme, and the therapeutic uses of ribozymes, are disclosed in Usman et al., Current Opin. Strict. Biol. 6:527-533 (1996). Ribozymes can also be prepared and used as described in Long et al., FASEB J. 7:25 (1993); Symons, Ann. Rev. Biochem. 61:641 (1992); Perrotta et al., Biochem. 31:16-17 (1992); Ojwang et al., PNAS 89:10802-10806 (1992); and U.S. Pat. No. 5,254,678.

Ribozyme cleavage of HIV-I RNA, methods of cleaving RNA using ribozymes, methods for increasing the specificity of ribozymes, and the preparation and use of ribozyme fragments in a hammerhead structure are described in U.S. Pat. Nos. 5,144,019; 5,116,742; and 5,225,337 and Koizumi et al., Nucleic Acid Res. 17:7059-7071 (1989). Preparation and use of ribozyme fragments in a hairpin structure are described by Chowrira and Burke, Nucleic Acids Res. 20:2835 (1992). Ribozymes can also be made by rolling transcription as described in Daubendiek and Kool, Nat. Biotechnol. 15(3):273-277 (1997).

The hybridizing region of the ribozyme may be modified or may be prepared as a branched structure as described in Horn and Urdea, Nucleic Acids Res. 17:6959-67 (1989). The basic structure of the ribozymes may also be chemically altered in ways familiar to those skilled in the art, and chemically synthesized ribozymes can be administered as synthetic oligonucleotide derivatives modified by monomeric units. In a therapeutic context, liposome mediated delivery of ribozymes improves cellular uptake, as described in Birikh et al., Eur. J. Biochem. 245:1-16 (1997).

Ribozymes of the present invention also include RNA endoribonucleases hereinafter “Cech-type ribozymes”) such as the one which occurs naturally in Tetrahymena thermophila (known as the IVS, or L-19 IVS RNA) and which has been extensively described by Thomas Cech and collaborators (Zaug et al., Science 224:574-578 (1984); Zaug and Cech, Science 231:470-475 (1986); Zaug et al., Nature, 324:429-433 (1986); WO 88/04300; Been and Cech, Cell 47:207-216 (1986)). The Cech-type ribozymes have an eight base pair active site which hybridizes to a target RNA sequence whereafter cleavage of the target RNA takes place. The invention encompasses those Cech-type ribozymes which target eight base-pair active site sequences that are present in a target gene.

Ribozymes can be composed of modified oligonucleotides (e.g., for improved stability, targeting, etc.) and should be delivered to cells which express the target gene in vivo. A preferred method of delivery involves using a DNA construct “encoding” the ribozyme under the control of a strong constitutive pol III or pol II promoter, so that transfected cells will produce sufficient quantities of the ribozyme to destroy endogenous messages and inhibit translation. Because ribozymes, unlike antisense molecules, are catalytic, a lower intracellular concentration is required for efficiency.

Using the nucleic acid sequences of the invention and methods known in the art, ribozymes are designed to specifically bind and cut the corresponding mRNA species. Ribozymes thus provide a means to inhibit the expression of any of the proteins encoded by the disclosed nucleic acids or their full-length genes. The full-length gene need not be known in order to design and use specific inhibitory ribozymes. In the case of a nucleic acid or cDNA of unknown function, ribozymes corresponding to that nucleotide sequence can be tested in vitro for efficacy in cleaving the target transcript. Those ribozymes that effect cleavage in vitro are further tested in vivo. The ribozyme can also be used to generate an animal model for a disease, as described in Birikh et al., Eur. J. Biochem. 245:1-16 (1997). An effective ribozyme is used to determine the function of the gene of interest by blocking its transcription and detecting a change in the cell. Where the gene is found to be a mediator in a disease, an effective ribozyme is designed and delivered in a gene therapy for blocking transcription and expression of the gene.

Therapeutic and functional genomic applications of ribozymes begin with knowledge of a portion of the coding sequence of the gene to be inhibited. Thus, for many genes, a partial nucleic acid sequence provides adequate sequence for constructing an effective ribozyme. A target cleavage site is selected in the target sequence, and a ribozyme is constructed based on the 5′ and 3′ nucleotide sequences that flank the cleavage site. Retroviral vectors are engineered to express monomeric and multimeric hammerhead ribozymes targeting the mRNA of the target coding sequence. These monomeric and multimeric ribozymes are tested in vitro for an ability to cleave the target mRNA. A cell line is stably transduced with the retroviral vectors expressing the ribozymes, and the transduction is confirmed by Northern blot analysis and reverse-transcription polymerase chain reaction (RT-PCR). The cells are screened for inactivation of the target mRNA by such indicators as reduction of expression of disease markers or reduction of the gene product of the target mRNA.

Cells and Organisms

Nucleic acid molecules that may be used in cell transformation or transfection may be any of the nucleic acid molecules of the present invention. Nucleic acid molecules of the present invention can be introduced into a cell or organism. In a preferred aspect, the cell is selected from the group consisting of cells that express very low levels of Her2, cells that express moderate levels of Her2, cells that express very high levels of Her2. In a more preferred aspect, the cell is a cancer cell, more preferably a cancer cell where Her2 is overexpressed relative to a non-transformed cell.

A host cell strain can be chosen for its ability to modulate the expression of the inserted sequences, to process an expressed reporter gene in the desired fashion, or based on the expression levels of an endogenous or heterologous Her2 gene. Mammalian cell lines available as hosts for expression are known in the art and include many immortalized cell lines available from the American Type Culture Collection (ATCC, Manassas, Va.), such as HeLa cells, Chinese hamster ovary (CHO) cells, baby hamster kidney (BHK) cells and a number of other cell lines. Non-limiting examples of suitable mammalian host cell lines include those shown below in Table 1.

TABLE 1
Mammalian Host Cell Lines

| Host Cell | Origin | Source |
|---|---|---|
| HepG-2 | Human Liver Hepatoblastoma | ATCC HB 8065 |
| CV-1 | African Green Monkey Kidney | ATCC CCL 70 |
| LLC-MK₂ | Rhesus Monkey Kidney | ATCC CCL 7 |
| 3T3 | Mouse Embryo Fibroblasts | ATCC CCL 92 |
| AV12-664 | Syrian Hamster | ATCC CRL 9595 |
| HeLa | Human Cervix Epitheloid | ATCC CCL 2 |
| RPMI8226 | Human Myeloma | ATCC CCL 155 |
| H4IIEC3 | Rat Hepatoma | ATCC CCL 1600 |
| C127I | Mouse Fibroblast | ATCC CCL 1616 |
| 293 | Human Embryonal Kidney | ATCC CRL 1573 |
| HS-Sultan | Human Plasma Cell Plasmocytoma | ATCC CCL 1484 |
| BHK-21 | Baby Hamster Kidney | ATCC CCL 10 |
| CHO-K1 | Chinese Hamster Ovary | ATCC CCL 61 |

Cell lines can be classified based on Her2 expression levels, for example without limitation, very low Her2 expressing cells can include PBMCs, foreskin fibroblast cells, U937 cells, and MDA-MB468 cells. The range of Her2 expression in very low Her2 expressing cells is about 0.0001 pgs of Her2/μg total protein to about 0.9 of Her2/μg total protein. More preferably, the range of Her2 expression in very low Her2 expressing cells is about 0.001 pgs of Her2/μg total protein to about 0.8 of Her2 μg total protein. In low Her2 expressing cells, the range of Her2 expression is about 1.0 pgs of Her2/μg total protein to about 15 pgs of Her2/μg total protein. More preferably, the range of Her2 expression in low Her2 expressing cells is about 1.2 pgs of Her2/μg total protein to about 8.0 of Her2/μg total protein. Low Her2 expressing cells can include, without limitation, HuH cells, 293T cells, and MCF-7 cells. In medium-high Her2 expressing cells, the range of Her2 expression is about 75 pgs of Her2/μg total protein to about 175 pgs of Her2/μg total protein. More preferably, the range of Her2 expression in medium-high Her2 expressing cells is about 110 pgs of Her2 μg total protein to about 150 of Her2 μg total protein. Medium-high Her2 expressing cells can include, without limitation, SKBR3 cells and BT474 cells.

In a preferred aspect, cells of the present invention can be cells of an organism. In a more preferred aspect, the organism is a mammal. In a most preferred aspect, the mammal is a human. In another more preferred aspect, the organism is a non-human mammal, preferably a mouse, rat, or a chimpanzee.

A nucleic acid of the present invention can be naturally occurring in the cell or can be introduced using techniques such as those described in the art. There are many methods for introducing transforming DNA segments into cells, but not all are suitable for delivering DNA to eukaryotic cells. Suitable methods include any method by which DNA can be introduced into a cell, such as by direct delivery of DNA, by desiccation/inhibition-mediated DNA uptake, by electroporation, by agitation with silicon carbide fibers, by acceleration of DNA coated particles, by chemical transfection, by lipofection or liposome-mediated transfection, by calcium chloride-mediated DNA uptake, etc. For example, without limitation, Lipofectamine® (Invitrogen Co., Carlsbad, Calif.) and Fugene® (Hoffmann-La Roche Inc., Nutley, N.J.) can be used for transfection of nucleic acid molecules, such as constructs and siRNA, into several mammalian cells. Alternatively, in certain embodiments, acceleration methods are preferred and include, for example, microprojectile bombardment and the like. Within the scope of this invention, the transfected nucleic acids of the present invention may be expressed transciently or stably. Such transfected cells can be in a two- or three-dimensional cell culture system or in an organism.

For example, without limitation, the construct may be an autonomously replicating construct, i.e., a construct that exists as an extrachromosomal entity, the replication of which is independent of chromosomal replication, e.g., a plasmid, an extrachromosomal element, a minichromosome, or an artificial chromosome. The construct may contain any means for assuring self-replication. For autonomous replication, the construct may further comprise an origin of replication enabling the construct to replicate autonomously in the host cell in question. Alternatively, the construct may be one which, when introduced into the cell, is integrated into the genome and replicated together with the chromosome(s) into which it has been integrated. This integration may be the result of homologous or non-homologous recombination.

Integration of a construct or nucleic acid into the genome by homologous recombination, regardless of the host being considered, relies on the nucleic acid sequence of the construct. Typically, the construct contains nucleic acid sequences for directing integration by homologous recombination into the genome of the host. These nucleic acid sequences enable the construct to be integrated into the host cell genome at a precise location or locations in one or more chromosomes. To increase the likelihood of integration at a precise location, there should be preferably two nucleic acid sequences that individually contain a sufficient number of nucleic acids, preferably 400 residues to 1500 residues, more preferably 800 residues to 1000 residues, which are highly homologous with the corresponding host cell target sequence. This enhances the probability of homologous recombination. These nucleic acid sequences may be any sequence that is homologous with a host cell target sequence and, furthermore, may or may not encode proteins.

Stable expression is preferred for long-term, high-yield production of recombinant proteins. For example, cell lines that stably express a reporter gene can be transformed using expression constructs that can contain viral origins of replication and/or endogenous expression elements and a selectable marker gene on the same or on a separate construct. Following the introduction of the construct, cells can be allowed to grow for 1-2 days in an enriched medium before they are switched to a selective medium. The purpose of the selectable marker is to confer resistance to selection, and its presence allows growth and recovery of cells that successfully express the introduced construct. Resistant clones of stably transformed cells can be proliferated using tissue culture techniques appropriate to the cell type. See, for example, ANIMAL CELL CULTURE, R. I. Freshney, ed., 1986.

Any number of selection systems can be used to recover transformed cell lines. These include, but are not limited to, the herpes simplex virus thymidine kinase (Wigler et al., 1977. Cell vol. 11:223-32) and adenine phosphoribosyltransferase (Lowy et al., 1980 Cell vol. 22:817-23.) genes which can be employed in tk⁻ or aprt⁻ cells, respectively. Also, antimetabolite, antibiotic, or herbicide resistance can be used as the basis for selection. For example, dhfr confers resistance to methotrexate (Wigler et al., 1980. Proc. Natl. Acad. Sci. vol. 77:3567-70), npt confers resistance to the aminoglycosides, neomycin and G-418 (Colbere-Garapin et al., 1981. J. Mol. Biol. vol. 150: 1-14), and als and pat confer resistance to chlorsulfuron and phosphinotricin acetyltransferase, respectively. Additional selectable genes have been described. For example, trpB allows cells to utilize indole in place of tryptophan, or hisD, which allows cells to utilize histinol in place of histidine (Hartman & Mulligan, 1988. Proc. Natl. Acad. Sci. vol. 85:8047-51). Visible markers such as anthocyanins, β-glucuronidase and its substrate GUS, and luciferase and its substrate luciferin, can be used to identify transformants and to quantify the amount of transient or stable protein expression attributable to a specific construct system (Rhodes et al., 1995. Methods Mol. Biol. vol. 55:121-131).

Although the presence of marker gene expression suggests that a reporter gene is also present, its presence and expression may need to be confirmed. For example, if a sequence encoding a reporter gene is inserted within a marker gene sequence, transformed cells containing sequences that encode a reporter gene can be identified by the absence of marker gene function. Alternatively, a marker gene can be placed in tandem with a sequence encoding a reporter gene under the control of a single promoter. Expression of the marker gene in response to induction or selection usually indicates expression of a reporter gene.

Alternatively, host cells which contain a reporter gene and which express a reporter gene e can be identified by a variety of procedures known to those of skill in the art. These procedures include, but are not limited to, DNA-DNA or DNA-RNA hybridizations and protein bioassay or immunoassay techniques that include membrane, solution, or chip-based technologies for the detection and/or quantification of nucleic acid or protein. For example, the presence of a reporter gene can be detected by DNA-DNA or DNA-RNA hybridization or amplification using probes or fragments or fragments of polynucleotides encoding a reporter gene. Nucleic acid amplification-based assays involve the use of oligonucleotides selected from sequences encoding a reporter gene to detect transformants that contain a reporter gene.

In a preferred embodiment, compounds of the present invention can include those with similar cell-specific effects, wherein the amount of modification observed in a SKBR-3 cell is greater than the amount of modification observed in a 293T cell. Similarly, breast cancer cells and cell lines may respond differently to compounds of the present invention. For example, in the presence of a compound of the present invention, the amount of modification observed in a HeLa cell can be less than the amount of modification observed in a BT-474 cell. A compound of the present invention is capable of producing a modification in translation to greater than a 5-fold increase over the 5′ her2 UTR in the absense of a 3′ her2 UTR or other TRE. As such, the cellular background, for which an indicator is the endogenous Her2 expression level, indicates the ability for regions of a 5′ her2 UTR, a 3′ her2 UTR, or both to modulate levels of reporter gene expression.

Polypeptides

Polypeptides of the invention may be identified using the screening methods described herein. By way of example, candidate polypeptides of the invention may be obtained from cancer cell lysates and purified using methods known in the art.

In a preferred embodiment, a specific polypeptide of the invention may be obtained from cancer cell lysates as follows. Total protein from cancer cell lysates are incubated with labeled RNA and UV-irratediated. After UV-crosslinking, unprotected areas of labeled RNA are digested with RNAse A, and the remaining labeled RNA molecules are resolved on SDS-PAGE to yield a 48-kDa polypeptide that specifically binds SEQ ID NO: 1 (see, e.g., example 10). The resulting 48-kDa polypeptide may then be further purified using methods known in the art (see, e.g., example 11). The screening methods of the present invention can then be used to confirm the ability of the 48-kDa polypeptide of the invention to modulate translational regulation by suppressing uORF-dependent repression of gene expression.

The 48-kDa polypeptide of the invention is expressed in all of the cancer cells studied, for examples without limitation 293T, HeLa, and HepG2, and expression has been shown to correlate with Her2 expression. For example, cell lines that over-express the Her2 protein also have a greater abundance of the 48-kDa polypeptide. Without intending to be limited by theory, it is believed that the 73-residue region from the Her2 3′ UTR is capable of recruiting the 48-kDa polypeptide. The presence of the 48-kDa polypeptide increases the interaction between the untranslated regions of the Her2 mRNA and the cellular translation machinery. Expression levels of the 48-kDa polypeptide contribute to Her2 over-expression, which is observed in a number of cancer cell lines.

Purification

Either naturally occurring or recombinant polypeptides of the present invention can be purified for use in assays of the present invention. Optionally, recombinant polypeptides are purified. Naturally occurring polypeptides are purified, e.g., from cancer cell lines such as SKBR3. Recombinant polypeptides are purified from any suitable bacterial or eukaryotic expression system, e.g., CHO cells or insect cells.

Polypeptides of the present invention may be purified to substantial purity by standard techniques, including selective precipitation with such substances as ammonium sulfate; column chromatography, immunopurification methods, and others (see, e.g., Scopes, Protein Purification: Principles and Practice (1982); U.S. Pat. No. 4,673,641; Ausubel et al., supra; and Sambrook et al., supra).

A number of procedures can be employed for purification. The polypeptides of the present invention can be separated from other polypeptides in cancer cell lysates by standard separation techniques well known to those of skill in the art. For example, polypeptides of the present invention can be purified using immunoaffinity columns, Sodium Dodecyl Sulfate-Polyacrylamide gel electrophoresis (SDS-PAGE), or a combination of methods well known to those of skill in the art. The 48-kDa polypeptide referred to herein migrates between 35-48 kDa on a 10-14% gradient gel (Bio-Rad Laboratories, Inc., Hercules, Calif.) when estimated by plotting the migration of the standards (Broad Range markers, Bio-Rad Laboratories, Inc., Hercules, Calif.) from the dye front as compared to the migration of the 48-kDa polypeptide.

In another example, polypeptides having established molecular adhesion properties, e.g. oligo-dT or biotin, can be associated with nucleic acids of the present invention. Biotinylated RNAs can be synthesized in vitro using Biotin-16-Uridine-5′-triphosphate (Hoffmann-La Roche Inc., Nutley, N.J.). With the appropriate ligand such as streptavidin, the modified nucleic acid of the present invention can be selectively bound to a purification column and then a polypeptide of the present invention can be isolated on the column in a relatively pure form. RNA affinity resin can be prepared by binding biotinylated RNAs to streptavidin-coated magnetic beads (Dynal-M280, Dynal ASA, Norway).

To isolate polypeptides of the present invention, cytoplasmic extracts from a breast cancer cell line (BT474 cells, for example) can be precleared using control affinity resin to remove non-specific RNA binding proteins and then incubated with a nucleic acid of the present invention. The resin is then washed extensively and the bound proteins are eluted with step-gradients of salt buffers. Fractions can be concentrated, dialyzed, or one or a combination of these methods can be used prior to subsequent purification steps or analysis such as is done with liquid chromatography (LC/MS) tandem mass spectrometry.

Standard Protein Separation Techniques for Purifying Polypeptides Solubility Fractionation

Often as an initial step, particularly if the protein mixture is complex, an initial salt fractionation can separate many of the unwanted host cell proteins (or proteins derived from the cell culture media) from the polypeptide of interest. The preferred salt is ammonium sulfate. Ammonium sulfate precipitates polypeptides by effectively reducing the amount of water in the protein mixture. Polypeptides then precipitate on the basis of their solubility. The more hydrophobic a polypeptide is, the more likely it is to precipitate at lower ammonium sulfate concentrations. A typical protocol includes adding saturated ammonium sulfate to a protein solution so that the resultant ammonium sulfate concentration is between 20-30%. This concentration will precipitate the most hydrophobic of polypeptides. The precipitate is then discarded (unless the protein of interest is hydrophobic) and ammonium sulfate is added to the supernatant to a concentration known to precipitate the protein of interest. The precipitate is then solubilized in buffer and the excess salt removed if necessary, either through dialysis or diafiltration. Other methods that rely on solubility of proteins, such as cold ethanol precipitation, are well known to those of skill in the art and can be used to fractionate complex protein mixtures.

Size Differential Filtration

The molecular weight of polypeptides can be used to isolate one from other polypeptides of greater and lesser size using ultrafiltration through membranes of different pore size (for example, Amicon or Millipore membranes). As a first step, the protein mixture is ultrafiltered through a membrane with a pore size that has a lower molecular weight cut-off than the molecular weight of the protein of interest. The retentate of the ultrafiltration is then ultrafiltered against a membrane with a molecular cut off greater than the molecular weight of the protein of interest. The recombinant protein will pass through the membrane into the filtrate. The filtrate can then be combined with other steps, for example chromatography as described below.

Column Chromatography

Polypeptides can also be separated from other polypeptides on the basis of size, net surface charge, hydrophobicity, and affinities. In addition, antibodies raised against polypeptides can be conjugated to column matrices and the polypeptides immunopurified. All of these methods are well known in the art. It will be apparent to one of skill that chromatographic techniques can be performed at any scale and using equipment from many different manufacturers (e.g., Pharmacia Biotech).

Methods of Action

Nucleic acid molecules of the present invention include nucleic acid molecules capable of recruiting one or more polypeptides of the present invention. Polypeptides included in the present invention include polypeptides that modify the number, frequency, or duration of interactions between the untranslated regions of Her-2 mRNA and the cellular translation machinery. Modification in the number, frequency, or duration of an interaction includes increases or decreases in one or a combination of these features that results in suppression of uORF-mediated repression. In a preferred embodiment, the uORF-mediated repression is the result of a 5′ her2 UTR. In a preferred embodiment, the uORF-mediated repression is cell-dependent. In a particularly preferred embodiment, the uORF-mediated repression is capable of being suppressed by a nucleic acid of the present invention in cancer cells, including without limitation SKBR-3 cells.

Small-molecules may be capable of inhibiting Her2 expression by modulating the expression of a polypeptide factor of the present invention. Alternatively, small-molecules may be capable of inhibiting Her2 expression by modulating the interactions of a polypeptide factor of the present invention with a nucleic acid of the present invention. In a preferred embodiment, the ability of a small-molecule to modulate the expression levels for specific nucleic acid molecules via a polypeptide factor of the present invention is cell-dependent. In a preferred embodiment, such specific nucleic acid molecules contain a uORF in their 5′ UTR. In a particularly preferred embodiment, a small-molecule modulates protein expression levels preferrentially in cancer cells by affecting one or more polypeptides of the present invention.

In a preferred embodiment, a polypeptide factor of the present invention can function as a proto-oncogene. The present invention is not limited by theory, but a polypeptide factor of the present invention includes a factor capable of up-regulating the translation of pro-oncogenic RNAs. In a particularly preferred invention, pro-oncogenic nucleic acid molecules that are normally poorly translated due to presence of uORFs in the molecule are up-regulated in the presence or absence of a polypeptide factor of the present invention. A polypeptide factor of the present invention can be linked to tumorigenesis due to its over-expression or activation in adult tumors. In a particulaly preferred embodiment, there is an increased expression of an approximately 48-kDa polypeptide factor in Her-2 over-expressing breast cancer cells. In a most preferred embodiment, expression of a polypeptide factor of the present invention is regulated at a post-transcriptional level. In another aspect, a polypeptide of the present invention can play a more global role in modulating translation, transport, stability, or a combination of such apects of expression through association with other polypeptides and nucleic acid sequences.

In another embodiment, polypeptides of the present invention are regulated via post-translational modifications. In a particularly preferred embodiment, the presence or absence of a post-translational modification of an approximately 48-kDa polypeptide factor is one mechanism of inhibiting Her-2 translation. In a most preferred embodiment, a quinazoline modulates a polypeptide factor of the present invention by effecting phosphorylation of the factor and phosphorylation results in the polypeptide regulating Her-2 expression.

In a preferred embodiment, a polypeptides of the present invention can include the following, without limitation: AUF1 (AU-rich RNA binding protein); int-6 (subunit of EIF3, eukaryotic translation initiation factor); HuR (regulator of mRNA stability and expression); and La protein (Lal A, Mazan-Mamczarz K, Kawai T, Yang X, Martindale J L, Gorospe M. (2004) EMBO J. August 4; 23(15):3092-102 and Asano K, Merrick W C, Hershey J W. (1997) J Biol Chem. September 19; 272(38):23477-80, are hereby incorporated by reference in their entirety.) In another preferred embodiment, AUF1, int-6, or both are not polypeptides of the present invention.

Screening Methods of the Present Invention

Compound

The present invention includes methods for screening compounds capable of modulating gene expression. Any compounds can be screened in an assay of the present invention.

In one aspect, a compound can be a nucleic acid or a non-nucleic acid, such as a polypeptide or a small-molecule. In a preferred embodiment, a nucleic acid can be a polynucleotide, a polynucleotide analog, a nucleotide, or a nucleotide analog. In a more preferred embodiment, a compound can be an antisense oligonucleotide, which are nucleotide sequences complementary to a specific DNA or RNA sequence of the present invention. Preferably, an antisense oligonucleotide is at least 11 nucleotides in length, but can be at least 12, 15, 20, 25, 30, 35, 40, 45, or 50 or more nucleotides long. Longer sequences also can be used. Antisense oligonucleotides can be deoxyribonucleotides, ribonucleotides, or a combination of both.

Nucleic acid molecules, including antisense oligonucleotide molecules, can be provided in a DNA construct and introduced into a cell. Nucleic acid molecules can be anti-sense or sense and double- or single-stranded. In a preferred embodiment, nucleic acid molecules can be interfering RNA (RNAi) or microRNA (mRNA). In a preferred embodiment, the dsRNA is 20-25 residues in length, termed small interfering RNAs (siRNA).

Oligonucleotides can be synthesized manually or by an automated synthesizer, by covalently linking the 5′ end of one nucleotide with the 3′ end of another nucleotide with non-phosphodiester internucleotide linkages such alkylphosphonates, phosphorothioates, phosphorodithioates, alkylphosphonothioates, alkylphosphonates, phosphoramidates, phosphate esters, carbamates, acetamidate, carboxymethyl esters, carbonates, and phosphate triesters. See Brown, 1994 Meth. Mol. Biol. vol. 20:1-8; Sonveaux, 1994. Meth. Mol. Biol. Vol. 26:1-72; and Uhlmann et al., 1990. Chem. Rev. vol. 90:543-583. Salts, esters, and other pharmaceutically acceptable forms of such compounds are also encompassed.

In an alternative embodiment, a compound can be a peptide, polypeptide, polypeptide analog, amino acid, or amino acid analog. Such a compound can be synthesized manually or by an automated synthesizer. In a preferred embodiment, the compound is a 48-kDa polypeptide factor that is capable of being UV-crosslinked to TRE1 in a physiological system.

In another embodiment, a compound can have a molecular weight less than about 10,000 grams per mole, less than about 5,000 grams per mole, less than about 1,000 grams per mole, less than about 500 grams per mole, less than about 100 grams per mole, and salts, esters, and other pharmaceutically acceptable forms of such compounds.

Compounds can be evaluated comprehensively for cytotoxicity. The cytotoxic effects of the compounds can be studied using cell lines, including for example 293T (kidney), HuH7 (liver), and Hela cells over about 4, 10, 16, 24, 36 or 72-hour periods. In addition, a number of primary cells such as normal fibroblasts and peripheral blood mononuclear cells (PBMCs) can be grown in the presence of compounds at various concentrations for about 4 days. Fresh compound can be added every other day to maintain a constant level of exposure with time. The effect of each compound on cell-proliferation can be determined by CellTiter 96® AQueous One Solution Cell Proliferation Assay (Promega Co, Madison, Wis.) and [³H]-Thymidine incorporation. Treatment of some cells with some of the compounds may have cytostatic effects. In a preferred aspect of the present invention, cytotoxicity of a compound in a cell correlates with the endogenous Her2 protein expression level of the cell. In a more preferred aspect, in very-low Her2 expressing cell lines, the CC₅₀ for a compound can be from about 90 μM to about 25 μM. In low and medium-high Her2 expressing cell lines, the CC₅₀ for a compound can be from about 20 μM to about 1 μM. In a most preferred aspect, in very-low Her2 expressing cell lines, the CC₅₀ for a compound can be from about 60 μm to about 25 μM. In low and medium-high Her2 expressing cell lines, the CC₅₀ for a compound can be from about 20 μM to about 1 μM. A selective index (ratios of CC₅₀ in cytotoxicity assays to the EC₅₀ in ELISA or FACS or the reporter gene assays) for each compound can be calculated for all of the UTR-reporters and protein inhibition assays. Compounds exhibiting substantial selective indices can be of interest and can be analyzed further in the functional assays.

Compounds can be pharmacologic agents already known in the art or can be compounds previously unknown to have any pharmacological activity. The compounds can be naturally occurring or designed in the laboratory. They can be isolated from microorganisms, animals, or plants, and can be produced recombinantly, or synthesized by chemical methods known in the art. If desired, compounds can be obtained using any of the numerous combinatorial library methods known in the art, including but not limited to, biological libraries, spatially addressable parallel solid phase or solution phase libraries, synthetic library methods requiring deconvolution, the “one-bead one-compound” library method, and synthetic library methods using affinity chromatography selection. Methods for the synthesis of molecular libraries are well known in the art (see, for example, DeWitt et al., Proc. Natl. Acad. Sci. U.S.A. 90, 6909, 1993; Erb et al. Proc. Natl. Acad. Sci. U.S.A. 91, 11422, 1994; Zuckermann et al., J. Med. Chem. 37, 2678, 1994; Cho et al., Science 261, 1303, 1993; Carell et al., Angew. Chem. Int. Ed. Engl. 33, 2059, 1994; Carell et al., Angew. Chem. Int. Ed. Engl. 33, 2061; Gallop et al., J. Med. Chem. 37, 1233, 1994). Libraries of compounds can be presented in solution (see, e.g., Houghten, BioTechniques 13, 412-421, 1992), or on beads (Lam, Nature 354, 82-84, 1991), chips (Fodor, Nature 364, 555-556, 1993), bacteria or spores (Ladner, U.S. Pat. No. 5,223,409), plasmids (Cull et al., Proc. Natl. Acad. Sci. U.S.A. 89, 1865-1869, 1992), or phage (Scott & Smith, Science 249, 386-390, 1990; Devlin, Science 249, 404-406, 1990); Cwirla et al., Proc. Natl. Acad. Sci. 97, 6378-6382, 1990; Felici, J. Mol. Biol. 222, 301-310, 1991; and Ladner, U.S. Pat. No. 5,223,409, the disclosures of which are hereby incorporated by reference.)

Particularly preferred compounds for use in screening assays of the present invention are wherein the compound is a quinazoline or quinoline, an inidazolopyridine, an indazole, or a derivative of any of these.

Screening Assays

The present invention includes and provides for assays capable of screening for compounds capable of modulating gene expression. In a preferred aspect of the present invention, an assay is an in vitro assay. In another aspect of the present invention, an assay is an in vivo assay. In a preferred aspect of the present invention, an assay measures translation. In another preferred aspect of the present invention, the assay includes a nucleic acid molecule of the present invention or a construct of the present invention or a polypeptide of the present invention. A nucleic acid molecule or construct of the present invention includes, without limitation, SEQ ID NO: 1, SEQ ID NOs: 7-22, or a sequence that differs from any of the residues in SEQ ID NO: 1 or SEQ ID NOs: 7-22 in that the nucleic acid sequence has been deleted, substituted, or added in a manner that does not alter the function. The present invention also provides fragments and complements of all the nucleic acid molecules of the present invention.

In a preferred aspect, a polypeptide of the present invention includes, without limitation, an approximately 48-kDa polypeptide factor capable of being UV-crosslinked to a nucleic acid molecule of the present invention. In a particularly preferred embodiment, a polypeptide of the present invention is capable of screening for other trans-acting or cis-acting polypeptides. Such screening assays are well known to those skilled in the art and include two-hybrid screens in yeast, expression libraries, and column chromatography using cellular lysates.

In one aspect of a preferred present invention, the activity or expression of a reporter gene is modulated. Modulated means increased or decreased during any point before, after, or during translation. In a preferred embodiment, activity or expression of a reporter gene is modulated during translation. For example, inhibition of translation of the reporter gene would modulate expression. In an alternative example, expression level of a reporter gene is modulated if the steady-state level of the expressed protein decreased even though translation was not inhibited. For example, a change in the half-life of an mRNA can modulate expression.

In an alternative embodiment, modulated activity or expression of a reporter gene means increased or decreased during any point before or during translation.

In a more preferred aspect, the activity or expression of a reporter gene or a target gene is modulated by greater than 50%, 60%, 70%, 80% or 90% in the presence of a compound. In a highly preferred aspect, more of an effect is observed in Her2-dependent cancer cells. In a particularly preferred aspect, Her2-dependent cancer cells can include medium-high Her2 expressing cells, including without limitation, SKBR3 cells, BT474 cells, and cells from a subject with cancer, such as Her2-dependent breast cancer cells from a mammal.

In a most preferred aspect, the activity or expression of a reporter gene is modulated without altering the activity of a control gene for general, indiscriminate translation activity. As used herein, indiscriminate translation activity refers to modulation in translation levels or activity that is random or unsystematic. One assay for modulation in general, indiscriminate translation activity uses a general translational inhibitor, for example puromycin, which is an inhibitor that causes release of nascent peptide and mRNA from ribosomes.

Expression of a reporter gene can be detected with, for example, techniques know in the art. Translation or transcription of a reporter gene can be detected in vitro or in vivo. In detection assays, either the compound or the reporter gene can comprise a detectable label, such as a fluorescent, radioisotopic, chemiluminescent, or enzymatic label, such as horseradish peroxidase, alkaline phosphatase, or luciferase. Detection of a compound, bound to an expressed reporter gene can then be accomplished, for example, by direct counting of radioemmission, by scintillation counting, or by determining conversion of an appropriate substrate to a detectable product.

High-throughput screening can be done by exposing nucleic acid molecules of the present invention to a library of compounds and detecting gene expression with assays known in the art, including, for example without limitation, those described above. In one embodiment of the present invention, cancer cells, such as MCF-7 cells, expressing a nucleic acid molecule of the present invention are treated with a library of compounds. Percent inhibition of reporter gene activity can be obtained with all the library compounds can be analyzed using, for example without limitation, a scattergram generated by SpotFiree (SpotFire, Inc., Somerville, Mass.). The high-throughput screen can be followed by subsequent selectivity screens. In a preferred embodiment, a subsequent selectivity screen can include detection of reporter gene expression in cells expressing, for example, a reporter gene linked to a 3′ her2 UTR variant or flanked by two 3′ her2 UTRs. In an alternate, preferred embodiment, a subsequent selectivity screen can include detection of reporter gene expression in cells in the presence of a various concentrations of compounds. In a particularly preferred embodiment, a screen can include detection of a polypeptide of the present invention in cells in the presence of a various concentrations of compounds.

A wide variety of labels and conjugation techniques are known by those skilled in the art and can be used in various nucleic acid and amino acid assays. Means for producing labeled hybridization or PCR probes for detecting sequences related to TREs of the present invention include oligolabeling, nick translation, end-labeling, or PCR amplification using a labeled nucleotide. Suitable reporter molecules or labels which can be used for ease of detection include radionuclides, enzymes, and fluorescent, chemiluminescent, or chromogenic agents, as well as substrates, cofactors, inhibitors, magnetic particles, and the like.

In another aspect of the present invention, the expression of a reporter gene is modulated by one or more cis-acting or trans-acting or a combination of both factors. Screening assays of the present invention include assays that monitor the presence or quantity of a polypeptide of the present invention. In a particularly preferred embodiment, translational up-regulation of Her-2 mRNA is dependent on an approximately 48-kDa polypeptide factor. In a most preferred embodiment, detection of the 48-kDa polypeptide can be used to predict or determine Her2 expression levels.

In Vitro

The present invention includes and provides for assays capable of screening for compounds capable of modulating gene expression. In a preferred aspect of the present invention, an assay is an in vitro assay. In a preferred aspect of the present invention, an in vitro assay that measures translation. In a preferred aspect of the present invention the in vitro assay includes a nucleic acid molecule of the present invention or a construct of the present invention.

In one embodiment, a reporter gene of the present invention can encode a fusion protein or a fusion protein comprising a domain that allows the expressed reporter gene to be bound to a solid support. For example, glutathione-5-transferase fusion proteins can be adsorbed onto glutathione sepharose beads (Sigma Chemical Co., St. Louis, Mo.) or glutathione derivatized microtiter plates, which are then combined with the compound or the compound and the non-adsorbed expressed reporter gene; the mixture is then incubated under conditions conducive to complex formation (e.g., at physiological conditions for salt and pH). Following incubation, the beads or microtiter plate wells are washed to remove any unbound components. Binding of the interactants can be determined either directly or indirectly, as described above. Alternatively, the complexes can be dissociated from the solid support before binding is determined.

Other techniques for immobilizing an expressed reporter gene or compound on a solid support also can be used in the screening assays of the invention. For example, either an expressed reporter gene or compound can be immobilized utilizing conjugation of biotin and streptavidin. Biotinylated expressed reporter genes or compounds can be prepared from biotin-NHS(N-hydroxysuccinimide) using techniques well known in the art (e.g., biotinylation kit, Pierce Chemicals, Rockford, Ill.) and immobilized in the wells of streptavidin-coated 96 well plates (Pierce Chemicals, Rockford, Ill.). Alternatively, antibodies which specifically bind to an expressed reporter gene or compound, but which do not interfere with a desired binding or catalytic site, can be derivatized to the wells of the plate. Unbound target or protein can be trapped in the wells by antibody conjugation.

Methods for detecting such complexes, in addition to those described above for the GST-immobilized complexes, include immunodetection of complexes using antibodies which specifically bind to an expressed reporter gene or compound, enzyme-linked assays which rely on detecting an activity of an expressed reporter gene, electrophoretic mobility shift assays (EMSA), and SDS gel electrophoresis under reducing or non-reducing conditions.

In one embodiment, translation of a reporter gene in vitro can be detected following the use of a reticulocyte lysate translation system, for example the TN® Coupled Reticulocyte Lysate System (Promega Co., Madison, Wis.). In this aspect, for example, without limitation, RNA (100 ng) can be translated at 30° C. in reaction mixtures containing 70% reticulocyte lysate, 20 μM amino acids and RNase inhibitor (0.8 units/μl). After 45 minutes of incubation, 20 μl of Luclite can be added and luminescence can be read on the View-Lux. Different concentrations of compounds can be added to the reaction in a final DMSO concentration of 2% and the EC₅₀ values calculated. Puromycin can be used as control for general indiscriminate translation inhibition. In vitro transcripts encoding a reporter gene linked to specific UTRs from target genes, including GAPDH, XIAP, TNF-α, and HIF-1α, can also be used.

To study the influence of cell-type specific factors, capped RNA can be translated in translation extracts prepared from specialized cells or cancer cell lines, for example without limitation, SKBR3 cells. Briefly, the cells can be washed with PBS and swollen in hypotonic buffer (10 mM Hepes, pH 7.4, 15 mM KCl, 1.5 mM Mg(OAc)₂, 2 mM DTT and 0.5 mM Pefabloc (Pentapharm Ltd. Co., Switzerland) for 5 minutes on ice. The cells can be lysed using a Dounce homogenizer (100 strokes), and the extracts can be spun for 10 min at 10,000×g. The clarified extracts can then be flash-frozen in liquid nitrogen and stored in aliquots at −70° C. Capped RNA (50 ng) in a reaction mixture containing 60% cellular translation extract, 15 μM total amino acids, 0.2 mg/ml Creatine phosho-kinase in 1× translation buffer (15 mM Hepes, pH 7.4, 85 mM KOAc, 1.5 mM Mg(OAc)₂, 0.5 mM ATP, 0.075 mM GTP, 18 mM creatine diphosphate and 1.5 mM DTT). After incubation of the translation reaction for 90 min at 37° C., activity of the protein encoded by the reporter gene can be detected. For activity of luciferase, encoded by the luciferase gene serving as the reporter gene, addition of 20 μl of LucLite® (Packard Instrument Co., Inc., Meriden, Conn.) can be used.

Capped and uncapped RNAs can be synthesized in vitro using the T7 polymerase transcription kits (Ambion Inc., Austin, Tex.). Capped RNAs from a variety of constructs, including constructs with Her2 linked to a TRE of the present invention, with a reporter gene linked only to a vector, with GAPDH linked to a TRE, with a HIF-1α linked to a TRE, and with a HIF-1α not linked to a TRE, can be used in a similar in vitro system to study the influence of cell-type specific factors on translation. In a preferred embodiment, such a vector contains a promoter functional in mammalian cells or bacteria or both.

In Vivo

The present invention includes and provides for assays capable of screening for compounds capable of modulating gene expression. In a preferred aspect of the present invention, an assay is an in vivo assay. Another preferred aspect of the present invention is an assay that measures translation. In a preferred aspect of the present invention, an in vivo assay includes a nucleic acid molecule of the present invention or a construct of the present invention.

In another embodiment, in vivo translation of a reporter gene can be detected. In a preferred embodiment, a reporter gene is transfected into a cancer cell obtained from a cell line available at the (American Type Culture Collection (ATCC), Manassas, Va.), for example HeLa, MCF-7, and COS-7, BT474. In a more preferred embodiment, a cancer cell has an altered genome relative to a similarly derived normal, primary cell, and the mammalian cancer cell proliferates under conditions where such a primary cell would not.

Screening for compounds that modulate reporter gene expression can be carried out in an intact cell. Any cell that comprises a reporter gene can be used in a cell-based assay system. A reporter gene can be naturally occurring in the cell or can be introduced using techniques such as those described above (see Cells and Organisms). In one embodiment, a cell line is chosen based on its expression levels of naturally occurring Her2. In a preferred embodiment, a cell line is chosen based on its moderate expression levels of naturally occurring Her2, for example the expression levels of naturally occurring Her2 in MCF-7 cells. the cell or can be introduced using techniques such as those described above. Modulation of reporter gene expression by a compound can be determined in vitro as described above or in vivo as described below.

To detect expression of endogenous protein, a variety of protocols for detecting and measuring the expression of a reporter gene are known in the art. For example, Enzyme-Linked Immunosorbent Assays (ELISAs), western blots using either polyclonal or monoclonal antibodies specific for an expressed reporter gene, Fluorescence-Activated Cell Sorter (FACS), electrophoretic mobility shift Assays (EMSA), or radioimmunoassay (RIA) can be performed to quantify the level of specific proteins in lysates derived from cells treated with the compounds.

A wide variety of labels and conjugation techniques are known by those skilled in the art and can be used in various nucleic acid and amino acid assays. Means for producing labeled hybridization or PCR probes for detecting sequences related to polynucleotides encoding PI-PLC-like enzyme polypeptides include oligolabeling, nick translation, end-labeling, or PCR amplification using a labeled nucleotide. Alternatively, sequences encoding a PI-PLC-like enzyme polypeptide can be cloned into a vector for the production of an mRNA probe. Such vectors are known in the art, are commercially available, and can be used to synthesize RNA probes in vitro by addition of labeled nucleotides and an appropriate RNA polymerase such as T7, T3, or SP6. These procedures can be conducted using a variety of commercially available kits (Amersham Biosciences Inc., Piscataway, N.J.; and Promega Co, Madison, Wis.). Suitable reporter molecules or labels which can be used for ease of detection include radionuclides, enzymes, and fluorescent, chemiluminescent, or chromogenic agents, as well as substrates, cofactors, inhibitors, magnetic particles, and the like.

Therapeutic Uses

The invention also provides pharmaceutical compositions that can be administered to a patient to achieve a therapeutic effect. Pharmaceutical compositions of the invention can comprise, for example, ribozymes or antisense oligonucleotides, antibodies that specifically bind to a TRE of the present invention, or mimetics, activators, inhibitors of a TRE activity, or a nucleic acid molecule of the present invention. The compositions can be administered alone or in combination with at least one other agent, such as stabilizing compound, which can be administered in any sterile, biocompatible pharmaceutical carrier, including, but not limited to, saline, buffered saline, dextrose, and water. The compositions can be administered to a patient alone, or in combination with other agents, drugs or hormones.

In addition to the active ingredients, these pharmaceutical compositions can contain suitable pharmaceutically-acceptable carriers comprising excipients and auxiliaries which facilitate processing of the active compounds into preparations which can be used pharmaceutically. Pharmaceutical compositions of the invention can be administered by any number of routes including, but not limited to, oral, intravenous, intramuscular, intra-arterial, intramedullary, intrathecal, intraventricular, transdermal, subcutaneous, intraperitoneal, intranasal, parenteral, topical, sublingual, or rectal means. Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

Pharmaceutical preparations for oral use can be obtained through combination of active compounds with solid excipient, optionally grinding a resulting mixture, and processing the mixture of granules, after adding suitable auxiliaries, if desired, to obtain tablets or dragee cores. Suitable excipients are carbohydrate or protein fillers, such as sugars, including lactose, sucrose, mannitol, or sorbitol; starch from corn, wheat, rice, potato, or other plants; cellulose, such as methyl cellulose, hydroxypropylmethyl-cellulose, or sodium carboxymethylcellulose; gums including arabic and tragacanth; and proteins such as gelatin and collagen. If desired, disintegrating or solubilizing agents can be added, such as the cross-linked polyvinyl pyrrolidone, agar, alginic acid, or a salt thereof, such as sodium alginate.

Pharmaceutical preparations which can be used orally include push-fit capsules made of gelatin, as well as soft, sealed capsules made of gelatin and a coating, such as glycerol or sorbitol. Push-fit capsules can contain active ingredients mixed with a filler or binders, such as lactose or starches, lubricants, such as talc or magnesium stearate, and, optionally, stabilizers. In soft capsules, the active compounds can be dissolved or suspended in suitable liquids, such as fatty oils, liquid, or liquid polyethylene glycol with or without stabilizers.

Pharmaceutical formulations suitable for parenteral administration can be formulated in aqueous solutions, preferably in physiologically compatible buffers such as Hanks' solution, Ringer's solution, or physiologically buffered saline. Aqueous injection suspensions can contain substances which increase the viscosity of the suspension, such as sodium carboxymethyl cellulose, sorbitol, or dextran. Additionally, suspensions of the active compounds can be prepared as appropriate oily injection suspensions. Suitable lipophilic solvents or vehicles include fatty oils such as sesame oil, or synthetic fatty acid esters, such as ethyl oleate or triglycerides, or liposomes. Non-lipid polycationic amino polymers also can be used for delivery. Optionally, the suspension also can contain suitable stabilizers or agents which increase the solubility of the compounds to allow for the preparation of highly concentrated solutions. For topical or nasal administration, penetrants appropriate to the particular barrier to be permeated are used in the formulation. Such penetrants are generally known in the art.

The pharmaceutical compositions of the present invention can be manufactured in a manner that is known in the art, e.g., by means of conventional mixing, dissolving, granulating, dragee-making, levigating, emulsifying, encapsulating, entrapping, or lyophilizing processes. The pharmaceutical composition can be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding free base forms. In other cases, the preferred preparation can be a lyophilized powder which can contain any or all of the following: 1-50 mM histidine, 0.1%-2% sucrose, and 2-7% mannitol, at a pH range of 4.5 to 5.5, that is combined with buffer prior to use. Further details on techniques for formulation and administration can be found in the latest edition of REMINGTON'S PHARMACEUTICAL SCIENCES (Maack Publishing Co., Easton, Pa.). After pharmaceutical compositions have been prepared, they can be placed in an appropriate container and labeled for treatment of an indicated condition. Such labeling would include amount, frequency, and method of administration.

Determination of a Therapeutically Effective Dose

A therapeutically effective dose refers to that amount of active ingredient which increases or decreases reporter gene activity relative to reporter gene activity which occurs in the absence of the therapeutically effective dose. For any compound, the therapeutically effective dose can be estimated initially either in cell culture assays or in animal models, usually mice, rabbits, dogs, or pigs. The animal model also can be used to determine the appropriate concentration range and route of administration. Such information can then be used to determine useful doses and routes for administration in humans.

Therapeutic efficacy and toxicity, e.g., ED₅₀ (the dose therapeutically effective in 50% of the population) and LD₅₀ (the dose lethal to 50% of the population), can be determined by standard pharmaceutical procedures in cell cultures or experimental animals. The dose ratio of toxic to therapeutic effects is the therapeutic index, and it can be expressed as the ratio, LD₅₀/ED₅₀.

Pharmaceutical compositions that exhibit large therapeutic indices are preferred. The data obtained from cell culture assays and animal studies is used in formulating a range of dosage for human use. The dosage contained in such compositions is preferably within a range of circulating concentrations that include the ED₅₀ with little or no toxicity. The dosage varies within this range depending upon the dosage form employed, sensitivity of the patient, and the route of administration.

The exact dosage will be determined by the practitioner, in light of factors related to the subject that requires treatment. Dosage and administration are adjusted to provide sufficient levels of the active ingredient or to maintain the desired effect. Factors which can be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions can be administered every 3 to 4 days, every week, or once every two weeks depending on the half-life and clearance rate of the particular formulation.

Normal dosage amounts can vary from 0.1 to 100,000 micrograms, up to a total dose of about 1 g, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc.

If the reagent is a single-chain antibody, polynucleotides encoding the antibody can be constructed and introduced into a cell either ex vivo or in vivo using well-established techniques including, but not limited to, transferrin-polycation-mediated DNA transfer, transfection with naked or encapsulated nucleic acids, liposome-mediated cellular fusion, intracellular transportation of DNA-coated latex beads, protoplast fusion, viral infection, electroporation, “gene gun,” and DEAE- or calcium phosphate-mediated transfection.

Effective in vivo dosages of an antibody are in the range of about 5 μg to about 50 μg/kg, about 50 μg to about 5 mg/kg, about 100 μg to about 500 μg/kg of patient body weight, and about 200 to about 250 μg/kg of patient body weight. For administration of polynucleotides encoding single-chain antibodies, effective in vivo dosages are in the range of about 100 ng to about 200 ng, 500 ng to about 50 mg, about 1 μg to about 2 mg, about 5 μg to about 500 μg, and about 20 μg to about 100 μg of DNA.

If the expression product is mRNA, the reagent is preferably an antisense oligonucleotide or a ribozyme. Polynucleotides that express antisense oligonucleotides or ribozymes can be introduced into cells by a variety of methods, as described above.

Preferably, a reagent reduces expression of a reporter gene or the activity of a reporter gene by at least about 10, preferably about 50, more preferably about 75, 90, or 100% relative to the absence of the reagent. The effectiveness of the mechanism chosen to decrease the level of expression of a reporter gene or the activity of a reporter gene can be assessed using methods well known in the art, such as hybridization of nucleotide probes to reporter gene-specific mRNA, quantitative RT-PCR, immunologic detection of an expressed reporter gene, or measurement of activity from an expressed reporter gene.

In any of the embodiments described above, any of the pharmaceutical compositions of the invention can be administered in combination with other appropriate therapeutic agents. Selection of the appropriate agents for use in combination therapy can be made by one of ordinary skill in the art, according to conventional pharmaceutical principles. The combination of therapeutic agents can act synergistically to effect the treatment or prevention of the various disorders described above. Using this approach, one may be able to achieve therapeutic efficacy with lower dosages of each agent, thus reducing the potential for adverse side effects.

Any of the therapeutic methods described above can be applied to any subject in need of such therapy, including, for example, mammals such as dogs, cats, cows, horses, rabbits, monkeys, and most preferably, humans.

Administration of a Therapeutically Effective Dose

A reagent which affects transcription or translation can be administered to a human cell, either in vitro or in vivo, to specifically reduce transcriptional or translational activity of a specific gene. In a preferred embodiment, the reagent preferably binds to a 5′ UTR of a gene. In an alternate embodiment, the present invention the reagent preferably binds to a TRE of the present invention. In a preferred embodiment, the reagent is a compound. For treatment of human cells ex vivo, an antibody can be added to a preparation of stem cells which have been removed from the body. The cells can then be replaced in the same or another human body, with or without clonal propagation, as is known in the art.

In one embodiment, the reagent is delivered using a liposome. Preferably, the liposome is stable in the animal into which it has been administered for at least about 30 minutes, more preferably for at least about 1 hour, and even more preferably for at least about 24 hours. A liposome comprises a lipid composition that is capable of targeting a reagent, particularly a polynucleotide, to a particular site in an animal, such as a human. Preferably, the lipid composition of the liposome is capable of targeting to a specific organ of an animal, such as the lung, liver, spleen, heart brain, lymph nodes, and skin.

A liposome useful in the present invention comprises a lipid composition that is capable of fusing with the plasma membrane of the targeted cell to deliver its contents to the cell. Preferably, the transfection efficiency of a liposome is about 0.5 μg of DNA per 16 nmole of liposome delivered to about 10⁶ cells, more preferably about 1.0 μg of DNA per 16 nmole of liposome delivered to about 10⁶ cells, and even more preferably about 2.0 μg of DNA per 16 nmol of liposome delivered to about 10⁶ cells. Preferably, a liposome is between about 100 and 500 nm, more preferably between about 150 and 450 nm, and even more preferably between about 200 and 400 nm in diameter.

Suitable liposomes for use in the present invention include those liposomes standardly used in, for example, gene delivery methods known to those of skill in the art. More preferred liposomes include liposomes having a polycationic lipid composition and/or liposomes having a cholesterol backbone conjugated to polyethylene glycol. Optionally, a liposome comprises a compound capable of targeting the liposome to a particular cell type, such as a cell-specific ligand exposed on the outer surface of the liposome.

Complexing a liposome with a reagent such as an antisense oligonucleotide or ribozyme can be achieved using methods that are standard in the art (see, for example, U.S. Pat. No. 5,705,151). Preferably, from about 0.1 μg to about 10 μg of polynucleotide is combined with about 8 nmol of liposomes, more preferably from about 0.5 μg to about 5 μg of polynucleotides are combined with about 8 nmol liposomes, and even more preferably about 1.0 μg of polynucleotides is combined with about 8 nmol liposomes.

In another embodiment, antibodies can be delivered to specific tissues in vivo using receptor-mediated targeted delivery. Receptor-mediated DNA delivery techniques are taught in, for example, Findeis et al. Trends in Biotechnol. 11, 202-05 (1993); Chiou et al., GENE THERAPEUTICS: METHODS AND APPLICATIONS OF DIRECT GENE TRANSFER (J. A. Wolff, ed.) (1994); Wu & Wu, J. Biol. Chem. 263, 621-24 (1988); Wu et al., J. Biol. Chem. 269, 542-46 (1994); Zenke et al., Proc. Natl. Acad. Sci. U.S.A. 87, 3655-59 (1990); Wu et al., J. Biol. Chem. 266, 338-42 (1991).

Diagnostic Methods

Agents of the present invention can also be used in diagnostic assays for detecting diseases and abnormalities or susceptibility to diseases and abnormalities related to the presence of mutations in the nucleic acid sequences that encode the TRE of the present invention. For example, differences can be determined between the cDNA or genomic sequence encoding a TRE in individuals afflicted with a disease and in normal individuals. If a mutation is observed in some or all of the afflicted individuals but not in normal individuals, then the mutation is likely to be the causative agent of the disease.

The present invention provides methods for detecting mutations at, at least, residues corresponding to position 513, 532, 533, or 534 of SEQ ID NO: 4 and nucleic acid molecules for use in detecting such mutations. Any nucleic acid molecule capable of detecting a mutation may be used and any method capable of detecting mutations can be adopted. Examples of suitable methods include, without limitation, hybridization assays such as northerns, RNAse protection assays and in situ hybridization. In a preferred method, the expression is compared by PCR-type assays. Assays and methods capable of detecting mutations at, at least, residues corresponding to position 513, 532, 533, or 534 of SEQ ID NO: 4 can be diagnostic or prognostic for the progression or treatment of Her2-related cancers.

For example, the direct DNA sequencing method can reveal sequence differences between a reference gene and a gene having mutations. In addition, cloned DNA segments can be employed as probes to detect specific DNA segments. The sensitivity of this method is greatly enhanced when combined with PCR. For example, a sequencing primer can be used with a double-stranded PCR product or a single-stranded template molecule generated by a modified PCR. The sequence determination is performed by conventional procedures using radiolabeled nucleotides or by automatic sequencing procedures using fluorescent tags.

Moreover, for example, genetic testing based on DNA sequence differences can be carried out by detection of alteration in electrophoretic mobility of DNA fragments in gels with or without denaturing agents. Small sequence deletions and insertions can be visualized, for example, by high-resolution gel electrophoresis. DNA fragments of different sequences can be distinguished on denaturing formamide gradient gels in which the mobilities of different DNA fragments are retarded in the gel at different positions according to their specific melting or partial melting temperatures (see, e.g., Myers et al., Science 230, 1242, 1985). Sequence changes at specific locations can also be revealed by nuclease protection assays, such as RNase and S1 protection or the chemical cleavage method (e.g., Cotton et al., Proc. Natl. Acad. Sci. USA 85, 4397-4401, 1985). Thus, the detection of a specific DNA sequence can be performed by methods such as hybridization, RNase protection, chemical cleavage, direct DNA sequencing or the use of restriction enzymes and Southern blotting of genomic DNA. In addition to direct methods such as gel-electrophoresis and DNA sequencing, mutations can also be detected by in situ analysis.

Altered levels of a TRE of the present invention can also be detected in various tissues. Assays used to detect levels of the receptor polypeptides in a body sample, such as blood or a tissue biopsy, derived from a host are well known to those of skill in the art and include radioimmunoassays, competitive binding assays, western blot analysis, and ELISA assays.

In another aspect of the present invention, agents of the present invention can also be used in diagnostic assays for detecting diseases and abnormalities or susceptibility to diseases and abnormalities related to the presence of mutations in or altered quantities of a polypeptide factor of the present invention. In a preferred embodiment, identification of a polypeptide factor of the present invention in a patient may facilitate classification of tumors and aid in selecting patient populations for designing a tailored cancer therapeutic treatment program.

In a particularly preferred aspect, assays to detect the level of a polypeptide factor or nucleic acid molecule of the present invention can be used in connection with other methods polypeptide factor of the present invention of staging and classification of tumors. In a most preferred embodiment, FISH analysis is used in combination with other methods used to detect polypeptides or nucleic acid molecules of the present invention in Her-2 positive tumors.

### EXAMPLES

Having now generally described the invention, the same will be more readily understood through reference to the following examples that are provided by way of illustration, and are not intended to be limiting of the present invention, unless specified.

### Example 1

### A 73-Residue Region from a 3′ her2 UTR Can Inhibit Translation of a Capped-5′+3′ her2 UTR Linked to a Luciferase Gene

A 73-residue region from a 3′ her2 UTR (SEQ ID NO: 1) and an N-terminal 310-residue region from a 3′ her2 UTR (SEQ ID NO: 5) are cloned into a construct downstream of a T7-promoter. RNA encoded for by these regions is synthesized using a T7-in vitro transcription kit (Ambion, Inc.; Austin, Tex.).

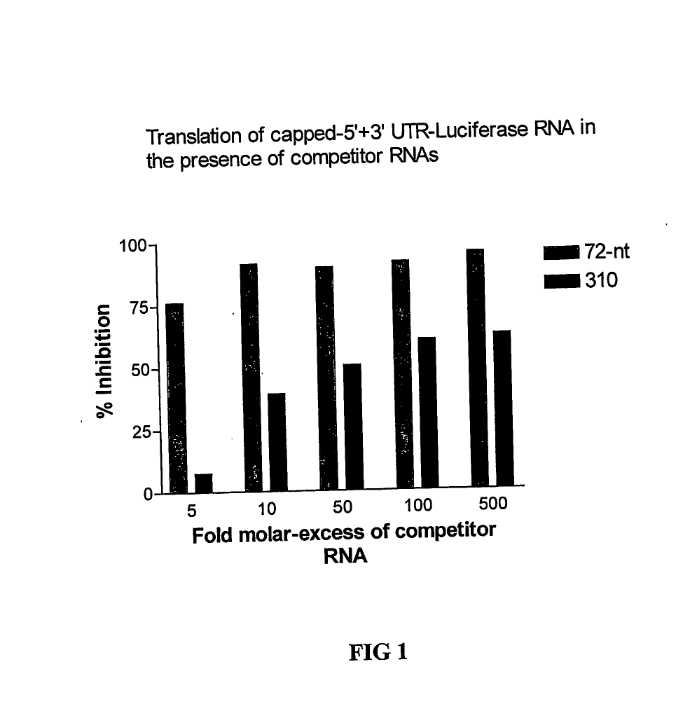

To quantitate the maximum level of translation, a capped-5′+3′ UTR-luciferase RNA (0.057 pmoles) is translated in an in vitro rabbit reticulocyte translation system for 60 minutes at 37° C. Then, a capped-5′+3′ UTR-luciferase RNA is translated in the presence of between a 5- to 500-fold molar excess of RNA from either the 73-residue region from a 3′ her2 UTR (SEQ ID NO: 1) or the N-terminal 310-residue region from a 3′ her2 UTR (SEQ ID NO: 5) under the same conditions. As shown in FIG. 1, translation of capped-5′+3′ UTR-luciferase RNA is inhibited about 75% in the presence of a 5-fold molar excess of the 73-residue region from a 3′ her2 UTR (SEQ ID NO: 1) relative to translation of capped-5′+3′ UTR-luciferase RNA in the absence of competitor RNA. Translation of capped-5′+3′ UTR linked to luciferase RNA is not significantly inhibited by about a 5-fold molar excess of the 310-residue region from a 3′ her2 UTR (SEQ ID NO: 5) relative to translation of capped-5′+3′ UTR-luciferase RNA in the absence of competitor RNA. The data presented shows that the 73-residue region from a 3′ her2 UTR (SEQ ID NO: 1), or a fragment thereof, has an ability to titrate out one or more factors required for efficient translation of RNA when the RNA is linked to a 5′ her2 UTR, or a 3′ her2 UTR, or both the 5′ and 3′ her2 UTRs.

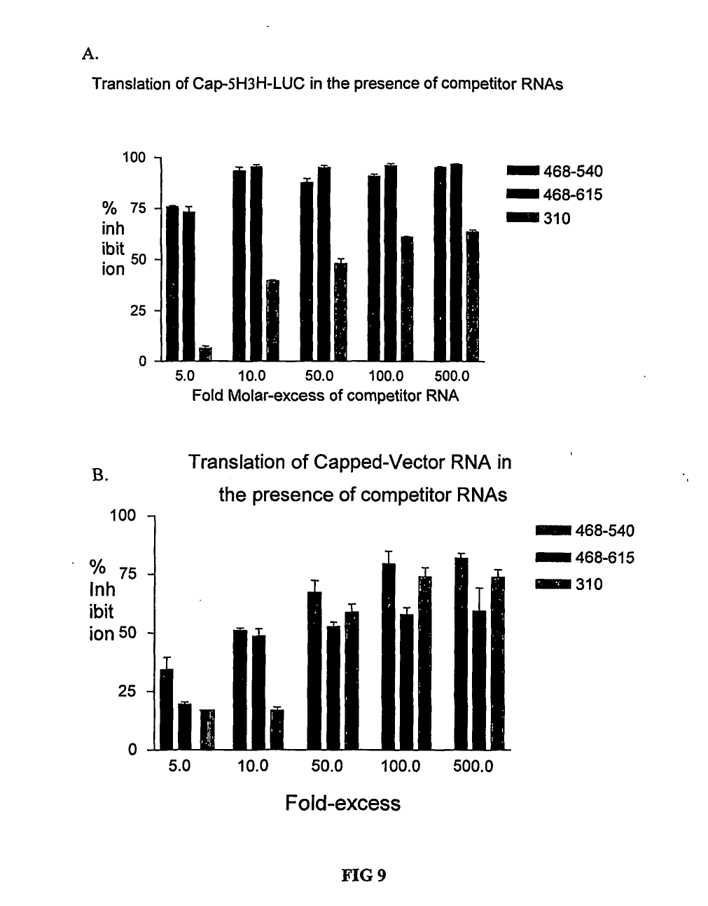

FIG. 9 shows results from similar in vitro experiments. Capped, in vitro-transcribed Her2 5′ UTR-luciferase-Her2 3′ UTR (5H3H-Luc) and vector only-luciferase (Vector-Luc) are translated in the presence or absence of competitor RNA fragments derived from a Her-2 3′ UTR. Nucleic acid molecules derived from nucleotides 468-540 of a Her2 3′ UTR (468-540) and from residues 468-615 of a Her2 3′ UTR (468-615) each contain the 73-nucleotide sequence (SEQ ID NO:1) essential for suppression of 5′ Her2 UTR repression. A Her2 3′ UTR fragment contains the first 310 nucleotides of the Her2 3′ UTR (1-310), but does not contain SEQ ID NO: 1. The percentage of inhibition of expression is less in the presence of the 1-310 fragment with respect to the percentage of inhibition of expression in the presence of either of the nucleic acid molecules containing SEQ ID NO: 1, 468-615 and 468-540. The percentage of inhibition for the 1-310 fragment is dose-dependent, whereas even at only a 5-fold molar excess of competitor RNA conatining SEQ ID NO: 1, expression is only at 25% of the maximum level of translation.

### Example 2

### Modulation of Protein Expression in Breast Cancer Cells

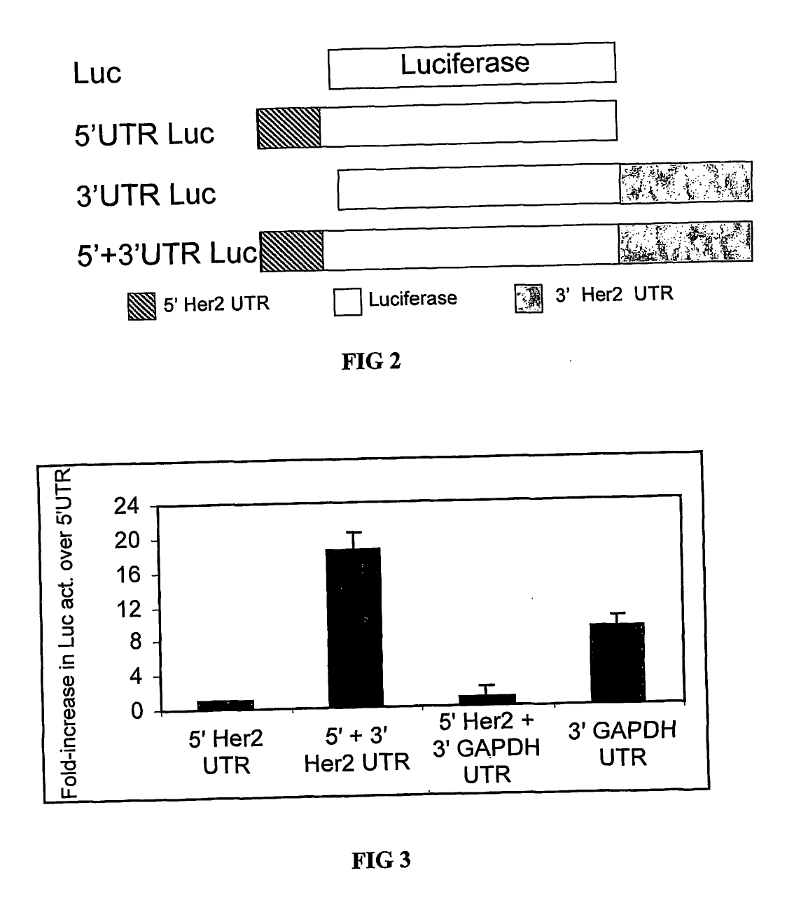

A luciferase reporter gene is linked to her2 UTRs to make various constructs (see FIG. 2). Each construct is transfected into a panel of cell-lines using Fugene® (Hoffmann-La Roche Inc., Nutley, N.J.), and the luciferase activity is measured 48 hours after transfection. The results are expressed as a fold-increase in luciferase activity over the repression caused by the 5′ her2 UTR linked to the luciferase reporter gene. A 5′ her2 UTR (SEQ ID NO: 6) represses translation of a luciferase reporter gene. Table 2 shows that in a number of cell lines, the 3′ her2 UTR linked to luciferase modulates the ability of the 5′ her2 UTR to inhibit translation. Table 2 shows that luciferase activity from MCF-7 cells transfected with luciferase RNA linked to a 3, her2 UTR is increased about 4-fold relative to the luciferase activity in MCF-7 cells transfected with a 5′ her2 UTR linked to the luciferase reporter gene. The greatest modulation is observed in the mammalian cancer cell lines that over-express Her2 protein, SKBR-3 and BT-474, about 13-fold and about 10-fold, respectively, showing that the efficiency of translation of a luciferase reporter gene mRNA in Her2 positive cells is regulated by interactions of regions in the 5′ her2 UTR and the 3′ her2 UTR. Moreover, the effect is more pronounced in high Her2 expressing cells relative to low Her2 expressing cell lines, especially for breast cancer cells.

TABLE 2
Fold Increase In Luciferase Activity Relative To Luciferase
Activity Repressed By A 5′ her2 UTR

| | Fold-increase over 5′ UTR |
|---|---|
| Cell Lines | Average |
| BT474 | 10 ± 4 |
| SKBR-3 | 13 ± 5 |
| MCF7 | 4 ± 2.2 |
| Hs578.1 | 2 ± 0.5 |
| HeLa | 2.23 ± 0.5 |
| 293T | 1.54 ± 1.0 |

One or more cellular factors play a role in regulating gene expression when the gene is linked to regions of a 5′ her2 UTR, a 3′ her2 UTR, or both. As shown in Table 2, the least modulation in luciferase activity was observed in 293T cells.

### Example 3

### A 3′ her2 UTR Specifically Overrides Translational Repression of a Reporter Gene Linked to a 5′ her2 UTR

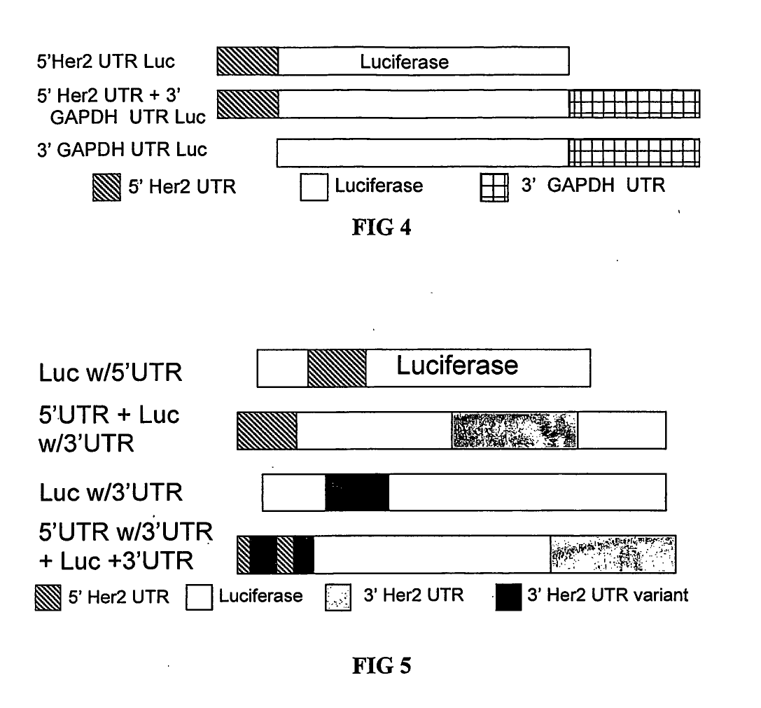

A 3′ her2 UTR (SEQ ID NO: 4) overrides the translational repression of a reporter gene linked to a 5′ her2 UTR (SEQ ID NO: 5) in mammalian cancer cells, specifically in SKBR3 cells. A 3′ UTR from a control gene, GAPDH, does not significantly modulate translational repression of a reporter gene linked to a 5′ her2 UTR. Constructs are generated that include a 5′ her2 UTR linked upstream of a luciferase gene that is linked upstream of a 3′ GAPDH UTR (see FIG. 4). Mammalian breast cancer cells are transfected with each of the constructs shown in FIG. 4 and, as shown in FIG. 3. The 3′ GAPDH UTR fails to overcome translational repression of a 5′ her2 UTR. Shown in FIG. 3, a luciferase gene linked upstream of a 3′ GAPDH UTR, but not linked to a 5′ her2 UTR, exhibits normal levels of translation.

### Example 4

### Detecting a Reporter Gene Expressed In Vivo

For detection of in vivo expression, cells are plated in a 6-well plate and treated with different concentrations of various compounds (for example, about 0.25, about 0.5, about 2.5, about 5 or about 10 μM) for about 4, about 10, about 24, about 36, or about 72 hours. At the end of a treatment, cells from each well are harvested and aliquots analyzed by FACS or ELISA or both. FACS analyses are performed using antibodies in various combinations. The FACS analysis involves determining the effect of known and unknown compounds on EGFR, Her3, and Her4 levels using labeled antibodies from BD Cytometry Systems (BD Biosciences, Canada). Control genes serve as negative controls and are quantified using specific antibodies (BD Biogen Pharmngen, Canada). Expression of control proteins should remain relatively constant under treatment with a compound with respect to the expression of a reporter protein. Monoclonal antibodies directed against such control proteins, for example the Na⁺, K⁺-ATPase and the integrin α6 subunit (CD49f; R&D Systems Minneapolis, Minn.) are used as negative controls. The Na⁺, K-ATPase, an integral membrane protein is ubiquitously expressed on the cell-surface. Integrin α6β4 is a structural component of hemidesmosomes and also functions as a receptor for laminin in stratified epithelia.

Lysates are prepared from cancer cells, such as BT474 cells, treated with different concentrations of compounds as described above and frozen at −20° C. Total protein concentration in the lysates are determined using the BCA protein assay reagent (Pierce Biotechnology, Rockford, Ill.). Cell-lysates are analyzed by ELISA, CC₅₀, and EC₅₀ values determined for various proteins, including for example Her2, EGFR, Her3, proliferating cell nuclear antigen (PCNA), and the cyclin D family. The effect of a compound on levels of PCNA is used to assist in determining whether a compound has any anti-proliferative effects in cancer cells. Specificity profiles of compounds can be obtained from ELISA protocols with standard techniques know in the art to determine expression of various proteins, including for example VEGF, XIAP, TNFα, GCSF, and Survivin.

### Example 5

### Effect of Deletions in a 3′ her2 UTR on Luciferase Expression

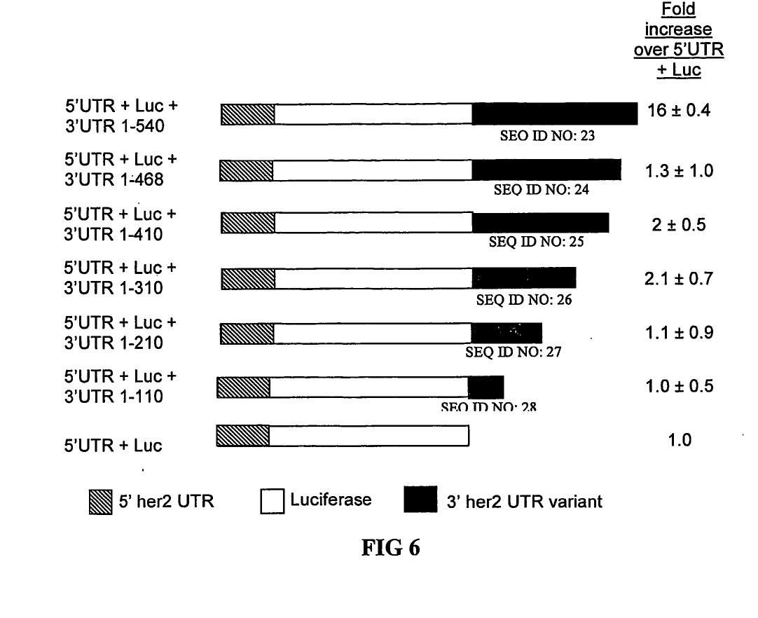

A luciferase reporter gene is linked to 3′ her2 UTR variants to make various constructs (see FIG. 6). Each construct is transfected into cell-lines, SKBR3 cells in particular, using Fugene® (Hoffmann-La Roche Inc., Nutley, N.J.), and the luciferase activity is measured 48 hours after transfection. The results are expressed as a fold-increase in luciferase activity over the repression caused by a 5′ her2 UTR (SEQ ID NO: 6) linked to the luciferase reporter gene (see FIG. 6). A 5′ her2 UTR represses translation of a reporter gene, such as luciferase.

Luciferase expression is increased about 17-fold (17±0.9) in a cell expressing a 5′ her2 UTR+Luc+3′her2 UTR with respect to a cell expressing a 5′ her2 UTR+Luc (see FIG. 5 for a schematic of the 5′ UTR Luc construct). Schematics for some constructs, including constructs with 3′ her2 UTR variants, are in FIG. 6. Of the constructs represented in FIG. 6, only cells expressing the construct that includes SEQ ID NO: 1 suppress the 5′ her2 UTR-mediated repression seen in cells expressing a 5′ her2 UTR+Luc construct. As such, suppression of the 5′ her2 UTR-mediated repression occurs when a 3′ her2 UTR contains SEQ ID NO: 1.

A 3′ her2 UTR comprising SEQ ID NO: 1 does not necessarily suppress 5′ her2 UTR-mediated repression. A 3′ her2 UTR with TRE23 (SEQ ID NO: 28) deleted, which is SEQ ID NO: 29, does not suppress 5′ her2 UTR-repressed protein expression. With a luciferase construct having a 3′ her2 UTR with TRE23 (SEQ ID NO: 28) deleted, expression of luciferase increases about 2-fold (1.7±0.9) over the expression of luciferase in the presence of only a 5′ her2 UTR (5′ her2 UTR+Luc).

Northern analysis of cell lines expressing a 3′ her2 UTR deletion or a 3′ her2 UTR variant or 3′ end mapping of the transcripts in such cells are used as controls for the experiments described above. Suppression of 5′ her2 UTR-mediated repression of luciferase by a 3′ her2 UTR deletion or variant is not due to a lack of expression or a change in the length of the transcripts.

### Example 6

### TRE1 Increases the Expression of Luciferase in Combination with a Ship-2 5′ UTR

Ship-2 mRNA contains a 33 nucleotide uORF (encoding 10 amino acids) that is located 14 nucleotides upstream of the main ORF. The uORF strongly represses translation of the main ORF.

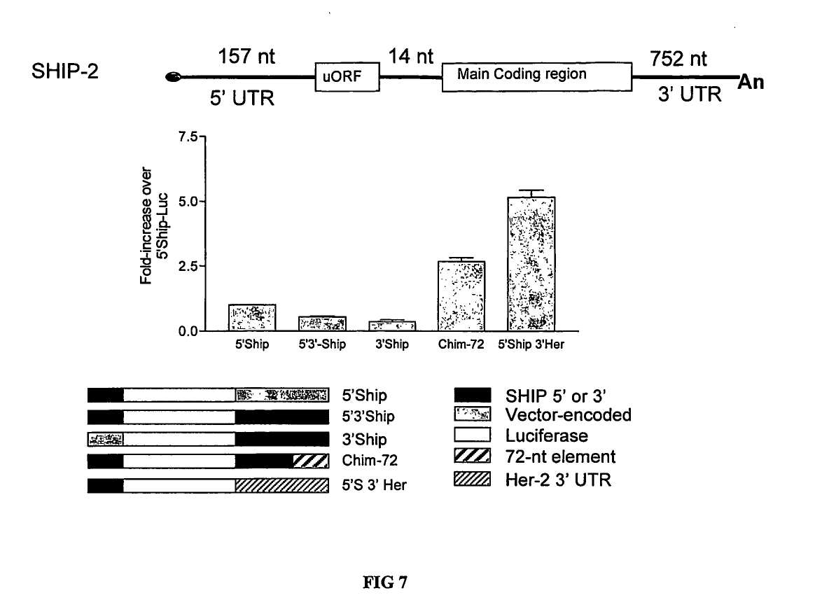

As shown in FIG. 7, the Her-23′ UTR overcomes translational repression by the Ship-2 uORF in SKBR3 cells. The presence of TRE1 in the 3′ UTR, when in combination with a Ship-2 5′ UTR, increases expression of luciferase about 5-fold relative to luciferase being flanked by 3′ and 5′ Ship-2 UTRs. Luciferase expression increases when flanked by a 5′ Ship-2 UTR and a chimeric 3′ UTR, consisting of 328 residues of Ship-2 3′ UTR flanked by the 5′ and 3′ end seqeunces of the Her-23′ UTR, relative to luciferase being flanked by 3′ and 5′ Ship-2 UTRs. When the chimeric 3′ UTR does not consist of TRE1, expression is reduced in comparison to in the presence of TRE1. The presence of the 3′ Her2 UTR residues 1-110 (SEQ ID NO: 28) in the chimeric 3′ UTR results in a slight increase in luciferase expression relative to 3′ Ship-2 UTR. Suppression of translational repression occurs in cells with chimeric constructs expressing a Ship-2 3′ UTR containing only TRE1 (SEQ ID NO: 1) from a Her-23′ UTR.

### Example 7

### her2 3′ UTR Effects Lucifierase Expression in the Presence of her2 uORF

Point mutations in the her2 5′ UTR are made using the QuikChange® mutagenesis kit (Stratagene; La Jolla, Calif.), and all of the constructs are confirmed by sequencing. Transient transfections are performed using Fugene® (Hoffmann-La Roche Inc., Nutley, N.J.) according to manufacturer's instructions. Briefly, SKBR3 cells are seeded at a density of 5×10⁵ cells per well of a 6-well plate and are grown for 48 hours until 80-90% confluency is obtained. Plasmid DNA (2 μg) is incubated with 6 μl Fugene® (Hoffmann-La Roche Inc., Nutley, N.J.) in 100 μl serum-free media for 15 minutes and the DNA-Fugene® (Hoffmann-La Roche Inc., Nutley, N.J.) complexes are added drop-wise to 2 ml serum containing media. After 12 hours, fresh media is added, and the cells are grown for another 48 hours.

Point mutagenesis at the start of the uORF in the her2 5′ UTR prevents a TRE1-dependent increase in protein expression level. When the lucifierase gene is in the presence of the her2 uORF and in the absense of TRE1 (“5′-Luc”), luciferase expression is low. When the lucifierase gene is in the presence of the her2 uORF and the TRE1 (“5′Luc3′”), luciferase expression is high, approximately 10-fold over 5′-Luc levels. There is no statistically significant difference in the luciferase expression between 5′Luc3′ and the luciferease expression when the luciferase gene is in the absense of the her2 uORF (uORF eliminated by ATG to AAG point mutation) and in the absense of TRE1 (“5H(ATG to AAG)”) or when the luciferase gene is in the absense of the her2 uORF and in the presence of TRE1 (“5′+3′ UTR (ATG to AAG)”). Relative to in 5′-Luc cells, luciferase expression in 5H (ATG to AAG) cells is approximately 11-fold greater, and luciferase expression in 5′+3′ UTR (ATG to AAG) cells is approximately 10-fold greater. Without the her2 uORF present, no effect of the 73-residue region is observed in the luciferase experiments. Point mutations in the her2 5′ UTR that alter the ATG of the uORF eliminate the ability of TRE1 (SEQ ID NO: 1) to increase protein expression levels.

### Example 8

### In Vitro Modulation of her2 UTR-Linked Reporter Gene Expression

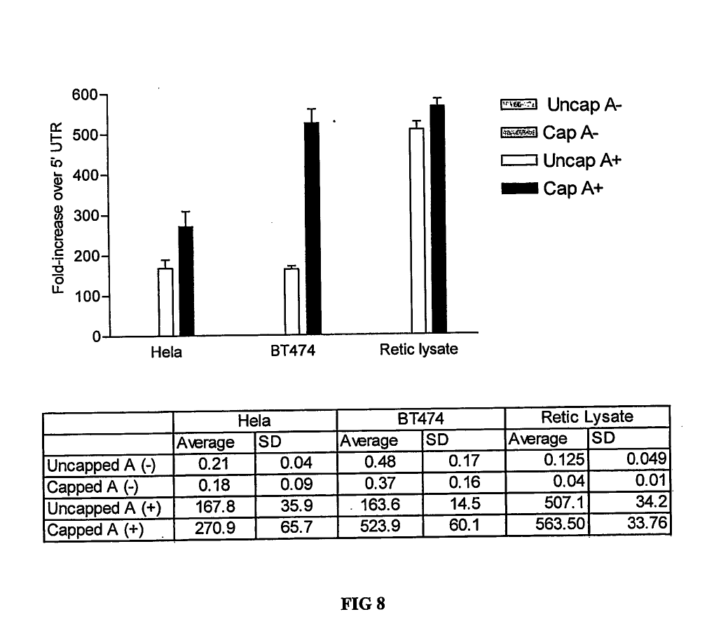

In vitro translation assays are performed with capped and uncapped RNAs in the presence or absence of 3′ poly(A) nucleic acid sequences. The 5′ UTR of her2 inhibits translation of the luciferase reporter gene in (1) retciculocyte lysates (2) Hela Extracts (3) BT474 cytoplasmic extracts. As shown in FIG. 8, there is a significant increase in translation of luciferase reporter expression in the presence of the her2 3′ UTR as compared to the expression in the presence of only the 5′ UTR of her2. Greater modulation of expression occurs for capped, poly(A+) RNA than for the uncapped, poly(A+) RNA in the in vitro systems when a 73-residue region (SEQ ID NO: 1) is linked to a 5′ her2 uORF in the luciferase experiments. The translation of 5′Her-Luc-3′Her mRNA occurs prefentially for poly(A) and cap-dependent molecules especially in BT474 extracts, a Her-2 over-expressing breast cancer cell-line.

Firefly-luciferase reporter activity is determined using the Bright-Glo™ Luciferase Assay System (Promega Corp.; Madison, Wis.). Total protein in each lysate is quantitated using the BCA micro-titer protein assay reagent (Pierce Biotechnology, Inc.; Rockford, Ill.). Luciferase activity is normalized to total protein content.

### Example 9

### Internal Initiation of Translation Based on UTR Sequence

To study if a GC-rich 5′ UTR or the regions of a 3′ UTR with secondary structure could facilitate internal initiation of translation, such sequences are cloned into a bi-cistronic vector (for example, p2Luci), and the constructs are transfected into SKBR3 cells. Cellular IRESs in VEGF, APAF and XIAP and the viral IRESs of HCV and EMCV are used as positive controls. There is no significant increase in Firefly Luc activity in bi-cistronic constructs with the Her-2 sequences or the weak IRES from APAF. The viral IRESs are capable of significantly increasing Firefly LUC translation in SKBR3 cells. XIAP is the only one, out of the three cellular IRESs, that functions in promoting internal initiation.

### Example 10

### Identification and Characterization of a 48-kDa Polypeptide a Trans-Acting Factor

Total protein from cancer cell lines is incubated with 10 fmoles of ³²P-UTP-labeled RNA for 1 hour at 37° C. in a final volume of 20 μl using Binding Buffer (20 mM Hepes-KOH, pH 7.5, 2.5 mM MgCl₂, 100 mM KCl, 20% glycerol, 0.5 mM dithiothreitol, protease inhibitor tablets). Reaction mixtures are UV-irradiated at 254 nm for 10 minutes with a StrataLinker® 11800 (Stratagene; La Jolla, Calif.) in Costar® 96-Well Cell Culture Clusters (CORNING COSTAR Co., Cambridge, Mass.) on ice. The reaction mixtures are then treated with RNAse A (2 mg/ml) for 30 minutes at 37° C. The samples are analyzed by 12% or 10-14% Criterion™ gels (Bio-Rad Laboratories, Inc., Hercules, Calif.) by Sodium Dodecyl Sulfate-Polyacrylamide gel electrophoresis (SDS-PAGE) and are detected by autoradiography.

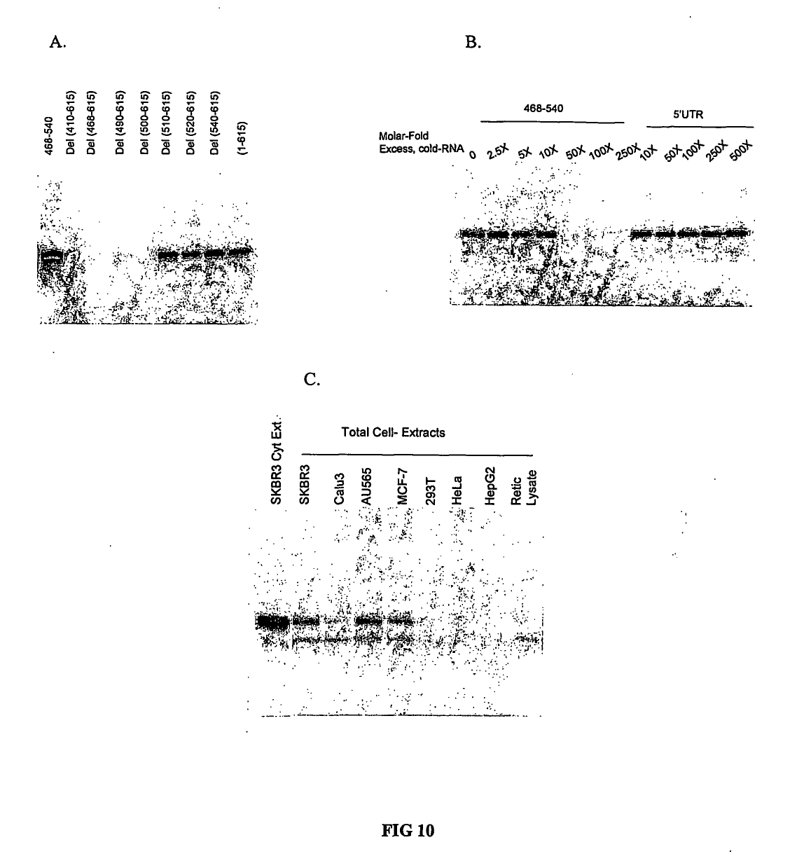

To study the role of trans-acting factors in modulating interactions between 5′ and 3′ UTRs, in vitro transcripts are synthesized, labeled, and incubated with cytoplasmic extracts from SKBR3 cells. After UV-crosslinking, unprotected areas of labeled RNA are digested with RNAse A, and the remaining labeled RNA molecules are resolved on SDS-PAGE. As shown in FIG. 10A, an approximately 48-kDa polypeptide crosslinks to a full-length Her2 3′ UTR (1-615) as well as to the 73-nucleotide element located between residues 468-540 of a 3′ Her2 UTR (SEQ ID NO:1).

The 48-kDa polypeptide does not crosslink to the RNA sequence from between residue 468 to residue 500 of a 3′ Her2 UTR and does not crosslink to the Her2 5′ UTR. Instead, a minimal binding region of 10 nucleotides, corresponding to the nucleotide sequence from residue 490 to residue 510 of a 3′ Her2 UTR, is essential for the 48-kDa polypeptide to UV-crosslink to a Her2 3′ UTR. Competition by a 50-fold molar excess of an unlabeled 73-nucleotide molecule (468-540; SEQ ID NO: 1), prevents binding of the 48-kDa polypeptide to the full-length, labeled Her2 3′ UTR. In comparison, competition with a 500-fold molar excess of nucleic acid molecules with sequences derived from the Her2 5′ UTR is ineffective in titrating out the 48-kDa polypepide. See FIG. 10B.

The 48-kDa polypeptide is expressed in all of the cancer cells studied, for examples without limitation 293T, HeLa, and HepG2. As shown in FIG. 10C, the relative abundance of the 48-kDa polypeptide correlates with Her2 expression, for example, cell lines that over-express the Her2 protein also have a greater abundance of the 48-kDa polypeptide. The 73-residue region from the Her2 3′ UTR is capable of recruiting the 48-kDa polypeptide. The presence of the 48-kDa polypeptide increases the interaction between the untranslated regions of the Her2 mRNA and the cellular translation machinery. Expression levels of the 48-kDa polypeptide contribute to Her2 over-expression, which is observed in a number of cancer cell lines.

### Example 11

### Purification of the 48-kDa Polypeptide

Biotinylated RNAs are synthesized in vitro using Biotin-16-Uridine-5′-triphosphate (Hoffmann-La Roche Inc., Nutley, N.J.). RNA affinity resin is prepared by binding biotinylated RNAs to streptavidin-coated magnetic beads (Dynal-M280, Dynal ASA, Norway). Two types of RNA-resins are prepared: RNA (1-410), which lacks the 73-nucleotide element (TRE1, SEQ ID NO: 1); and RNA (1-540), which contains the 73-nucleotide element (TRE1, SEQ ID NO: 1). Cytoplasmic extract (about 4 ml) from a breast cancer cell line (BT474 cells, for example) is precleared using the affinity resin RNA (1-410) to remove non-specific RNA binding proteins. After preclearing, the unbound proteins are incubated with RNA containing the 73-nucleotide element (TRE1, SEQ ID NO: 1). The resin is then washed extensively and the bound proteins are eluted with step-gradients in buffers containing 0.2M, 2M, and 4M of salt. Then, the fractions are concentrated and dialyzed. The activity in each fraction is determined using a UV-crosslinking assay as described above. The band corresponding to the UV-crosslinked band is identified and sequence analysis is done using LC/MS tandem mass spectrometry.

Each periodical, patent, and other document or reference cited herein is herein incorporated by reference in its entirety.

What is claimed:

1. A method of determining whether a candidate compound modulates gene expression, comprising:
(a) providing a compound and a reporter gene in a system, wherein said reporter gene linked to an untranslated region comprising SEQ ID NO: 1; and
(b) detecting expression of said reporter gene in said system, wherein expression of said reporter gene is altered relative to expression of a reporter gene not linked to an untranslated region comprising SEQ ID NO: 1.

2. The method according to claim 1, wherein said untranslated region comprising SEQ ID NO: 1 is downstream of said reporter gene.

3. The method according to claim 2, wherein said untranslated region comprising SEQ ID NO: 1 is between about 1000 to about 500 residues upstream from the 5′ end of a mRNA poly(A) tail.

4. The method according to claim 2, wherein said untranslated region comprising SEQ ID NO: 1 is between about 500 to about 100 residues upstream from the 5′ end of a mRNA poly(A) tail.

5. The method according to claim 2, wherein said untranslated region comprising SEQ ID NO: 1 is between about 100 to about 60 residues upstream from the 5′ end of a mRNA poly(A) tail.

6. The method according to claim 2, wherein said untranslated region comprising SEQ ID NO: 1 is about 80 residues upstream from the 5′ end of a mRNA poly(A) tail.

7. The method according to claim 1, wherein an upstream open reading frame (uORF) is upstream of said reporter gene.

8. The method according to claim 1, wherein said reporter gene not linked to an untranslated region comprising SEQ ID NO: 1 is a reporter gene linked to an untranslated region from a control gene.

9. The method according to claim 1, wherein said expression of said reporter gene not linked to an untranslated region comprising SEQ ID NO: 1 is greater than zero.

10. The method according to claim 1, wherein said expression of said reporter gene not linked to an untranslated region comprising SEQ ID NO: 1 is greater than said expression of said reporter gene linked to an untranslated region comprising SEQ ID NO: 1.

11. The method according to claim 1, wherein said expression of said reporter gene not linked to an untranslated region comprising SEQ ID NO: 1 is less than said expression of said reporter gene linked to an untranslated region comprising SEQ ID NO: 1.

12. The method according to claim 1, wherein said reporter gene is located within a cell.

13. The method according to claim 12, wherein said cell is a mammalian cell.

14. The method according to claim 13, wherein said mammalian cell is a mammalian cancer cell.

15. The method according to claim 14, wherein said mammalian cancer cell is a MCF-7 cell.

16. The method according to claim 14, wherein said mammalian cancer cell is a Her2 overexpressing mammalian breast cancer cell.

17. The method according to claim 16, wherein said Her2 overexpressing mammalian breast cancer cell is a BT474 cell.

18. The method according to claim 1, wherein said reporter gene is translated in vitro.

19. The method according to claim 18, wherein said reporter gene is translated in the presence of a cellular extract.

20. The method according to claim 1, wherein said compound is a small-interfering RNA molecule.

21. A method of determining whether a candidate compound modulates gene expression, comprising:
(a) providing a reporter gene linked to an untranslated region from a target gene and a compound, wherein said untranslated region from a target gene is linked to SEQ ID NO: 1;
(b) detecting expression of said linked reporter gene;
(c) providing a reporter gene not linked to an untranslated region comprising SEQ ID NO: 1 and a compound; and
(d) detecting expression of said not linked reporter gene.

22. The method according to claim 21, wherein said untranslated region comprising SEQ ID NO: 1 is downstream of said reporter gene.

23. The method according to claim 21, wherein an uORF is upstream of said reporter gene.

24. The method according to claim 21, further comprising:
(e) comparing said expression of said linked reporter gene to said expression of said unlinked reporter gene.

25. A method comprising:
(a) providing a reporter gene linked to an untranslated region from a target gene and a compound, wherein said untranslated region from a target gene is linked to SEQ ID NO: 1; and
(b) detecting expression of said reporter gene, wherein said expression of said reporter gene is greater relative to expression of a reporter gene not linked to SEQ ID NO: 1.

26. The method according to claim 25, further comprising:
(c) detecting expression of said reporter gene not linked to SEQ ID NO: 1.

27. The method according to claim 25, further comprising:
(d) comparing said expression of said linked reporter gene to said expression of said not linked reporter gene.

28. A cell line comprising a reporter gene linked to an untranslated region comprising SEQ ID NO: 1.

29. The cell line according to claim 28, wherein said reporter gene is expressed at a level within an order of magnitude relative to a cell line comprising a reporter gene not linked to said untranslated region comprising SEQ ID NO: 1.

30. The cell line according to claim 28, wherein said reporter gene is stably expressed for six months or more.

31. The cell line according to claim 30, wherein said cell line is derived from a MCF-7 cell line.

32. A hybrid comprising a nucleic acid molecule comprising SEQ ID NO: 1 and a compound, wherein said compound is capable of inhibiting expression of a reporter gene linked to said nucleic acid molecule comprising SEQ ID NO: 1 relative to expression of a reporter gene not linked to a nucleic acid molecule comprising SEQ ID NO: 1.

33. The hybrid of claim 32, wherein the EC₅₀ value of said compound is 5-20 fold less for expression of a reporter gene linked to an untranslated region comprising SEQ ID NO: 1 than for expression of a reporter gene not linked to an untranslated region comprising SEQ ID NO: 1.

34. The hybrid of claim 32, wherein the specificity of said compound is greater for a nucleic acid molecule comprising SEQ ID NO: 1 than for a nucleic acid molecule not comprising SEQ ID NO: 1.

35. The hybrid of claim 32, wherein the selectivity of said compound is at least ten-fold greater for a nucleic acid molecule comprising SEQ ID NO: 1 than for a nucleic acid molecule not comprising SEQ ID NO: 1.

36. The hybrid of claim 32, wherein said compound does not inhibit the activity of a protein encoded for by said reporter gene linked to said nucleic acid molecule comprising SEQ ID NO: 1

37. The hybrid of claim 32, wherein said nucleic acid molecule comprising SEQ ID NO: 1 is a RNA molecule.

38. The hybrid of claim 32, wherein said compound inhibits expression of said reporter gene linked to said nucleic acid molecule comprising SEQ ID NO: 1 in a Her2 overexpressing breast cancer cell more than it inhibits expression of said reporter gene linked to an untranslated region comprising SEQ ID NO: 1 in a MCF-7 cell.

39. The hybrid of claim 38, wherein said Her2 overexpressing breast cancer cell is a BT474 cell.

40. The hybrid of claim 32, wherein said compound is a nucleic acid molecule.

41. The hybrid of claim 32, wherein said compound is a quinazoline or quinoline or a derivative thereof either.

42. The hybrid of claim 32, wherein said compound is an inidazolopyridine or a derivative thereof.

43. The hybrid of claim 32, wherein said compound is an indazole or a derivative thereof.

44. A hybrid of a compound and a nucleic acid molecule comprising SEQ ID NO: 1, wherein said compound is capable of preferentially binding said nucleic acid molecule relative to a nucleic acid molecule not comprising SEQ ID NO: 1.

45. A substantially purified nucleic acid molecule comprising between 95% and 99% sequence identity with a nucleic acid molecule of SEQ ID NO: 1, a fragment thereof, or a complement of either.

46. The substantially purified nucleic acid molecule according to claim 45 that modulates expression of a gene selected from the group consisting of Mdm-2, Ship-2, Estrogen-receptor-α, S-AdoMet, CCAAT/Enhancer-binding protein-α, and CCAAT/Enhancer-binding protein-β.

47. A substantially purified nucleic acid molecule consisting of SEQ ID NO: 1, a fragment thereof, or a complement of either.

48. The substantially purified nucleic acid molecule according to claim 47 that modulates expression of a gene selected from the group consisting of Mdm-2, Ship-2, Estrogen-receptor-α, S-AdoMet, CCAAT/Enhancer-binding protein-α, and CCAAT/Enhancer-binding protein-β.

49. A method for identifying a compound that modulates reporter gene expression comprising:
(a) providing a reporter gene linked to an untranslated region comprising SEQ ID NO: 1 and a cellular extract; and
(b) detecting expression of said reporter gene, wherein said compound modulates expression of said reporter gene relative to expression of a reporter gene not linked to an untranslated region comprising SEQ ID NO: 1.

50. The method of claim 49, wherein said cellular extract is from a cancer cell.

51. The method of claim 50, wherein said cancer cell is a Her2 overexpressing cancer cell.

52. The method of claim 51, wherein said Her2 overexpressing cancer cell is a BT474 cell.

53. A substantially purified polypeptide characterized by:
(a) a molecular weight of approximately 48-kDa on a 10-14% gradient SDS-polyacrylamide gel electrophoresis (SDS-PAGE);
(b) its ability to specifically bind SEQ ID NO: 1;
(c) its ability to suppresses uORF-dependent repression of gene expression; and
(d) its ability to cross-link to a Her2 3′ UTR under physiological conditions.

54. The substantially purified polypeptide of claim 53, wherein said polypepide expression is regulated by a kinase.


SEQUENCE LISTING (30 sequences)

SEQ ID NO: 1 - DNA, 73 bp, Artificial
  Synthetic construct
cttttctgtttagtttttactttttttgttttgtttttttaaagacgaaataaagacccaggggagaatgggt

SEQ ID NO: 2 - DNA, 3768 bp, Homo sapiens
atggagctggcggccttgtgccgctgggggctcctcctcgccctcttgccccccggagccgcgagcacccaagtgtgcaccggcacagacatgaagctgcggctccctgccagtcccgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaaggacatcttccacaagaacaaccagctggctctcacactgatagacaccaaccgctctcgggcctgccacccctgttctccgatgtgtaagggctcccgctgctggggagagagttctgaggattgtcagagcctgacgcgcactgtctgtgccggtggctgtgcccgctgcaaggggccactgcccactgactgctgccatgagcagtgtgctgccggctgcacgggccccaagcactctgactgcctggcctgcctccacttcaaccacagtggcatctgtgagctgcactgcccagccctggtcacctacaacacagacacgtttgagtccatgcccaatcccgagggccggtatacattcggcgccagctgtgtgactgcctgtccctacaactacctttctacggacgtgggatcctgcaccctcgtctgccccctgcacaaccaagaggtgacagcagaggatggaacacagcggtgtgagaagtgcagcaagccctgtgcccgagtgtgctatggtctgggcatggagcacttgcgagaggtgagggcagttaccagtgccaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcactgctggggtccagggcccacccagtgtgtcaactgcagccagttccttcggggccaggagtgcgtggaggaatgccgagtactgcaggggctccccagggagtatgtgaatgccaggcactgtttgccgtgccaccctgagtgtcagccccagaatggctcagtgacctgttttggaccggaggctgaccagtgtgtggcctgtgcccactataaggaccctcccttctgcgtggcccgctgccccagcggtgtgaaacctgacctctcctacatgcccatctggaagtttccagatgaggagggcgcatgccagccttgccccatcaactgcacccactcctgtgtggacctggatgacaagggctgccccgccgagcagagagccagccctctgacgtccatcgtctctgcggtggttggcattctgctggtcgtggtcttgggggtggtctttgggatcctcatcaagcgacggcagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagaccatgtccgggaaaaccgcggacgcctgggctcccaggacctgctgaactggtgtatgcagattgccaaggggatgagctacctggaggatgtgcggctcgtacacagggacttggccgctcggaacgtgctggtcaagagtcccaaccatgtcaaaattacagacttcgggctggctcggctgctggacattgacgagacagagtaccatgcagatgggggcaaggtgcccatcaagtggatggcgctggagtccattctccgccggcggttcacccaccagagtgatgtgtggagttatggtgtgactgtgtgggagctgatgacttttggggccaaaccttacgatgggatcccagcccgggagatccctgacctgctggaaaagggggagcggctgccccagccccccatctgcaccattgatgtctacatgatcatggtcaaatgttggatgattgactctgaatgtcggccaagattccgggagttggtgtctgaattctcccgcatggccagggacccccagcgctttgtggtcatccagaatgaggacttgggcccagccagtcccttggacagcaccttctaccgctcactgctggaggacgatgacatgggggacctggtggatgctgaggagtatctggtaccccagcagggcttcttctgtccagaccctgccccgggcgctgggggcatggtccaccacaggcaccgcagctcatctaccaggagtggcggtggggacctgacactagggctggagccctctgaagaggaggcccccaggtctccactggcaccctccgaaggggctggctccgatgtatttgatggtgacctgggaatgggggcagccaaggggctgcaaagcctccccacacatgaccccagccctctacagcggtacagtgaggaccccacagtacccctgccctctgagactgatggctacgttgcccccctgacctgcagcccccagcctgaatatgtgaaccagccagatgttcggccccagcccccttcgccccgagagggccctctgcctgctgcccgacctgctggtgccactctggaaagggccaagactctctccccagggaagaatggggtcgtcaaagacgtttttgcctttgggggtgccgtggagaaccccgagtacttgacaccccagggaggagctgcccctcagccccaccctcctcctgccttcagcccagccttcgacaacctctattactgggaccaggacccaccagagcggggggctccacccagcaccttcaaagggacacctacggcagagaacccagagtacctgggtctggacgtgccagtgtga

SEQ ID NO: 3 - DNA, 531 bp, Artificial
  Synthetic construct
accagaaggccaagtccgcagaagccctgatgtgtcctcagggagcagggaaggcctgacttctgctggcatcaagaggtgggagggccctccgaccacttccaggggaacctgccatgccaggaacctgtcctaaggaaccttccttcctgcttgagttcccagatggctggaaggggtccagcctcgttggaagaggaacagcactggggagtctttgtggattctgaggccctgcccaatgagactctagggtccagtggatgccacagcccagcttggccctttccttccagatcctgggtactgaaagccttagggaagctggcctgagaggggaagcggccctaagggagtgtctaagaacaaaagcgacccattcagagactgtccctgaaacctagtactgccccccatgaggaaggaacagcaatggtgtcagtatccaggctttgtacagagtgcttttctgtttagtttttactttttttgttttgtttttttaaagatgaaataaagacccagggggag

SEQ ID NO: 4 - DNA, 615 bp, Artificial
  Synthetic construct
tgaaccagaaggccaagtccgcagaagccctgatgtgtcctcagggagcagggaaggcctgacttctgctggcatcaagaggtgggagggccctccgaccacttccaggggaacctgccatgccaggaacctgtcctaaggaaccttccttcctgcttgagttcccagatggctggaaggggtccagcctcgttggaagaggaacagcactggggagtctttgtggattctgaggccctgcccaatgagactctagggtccagtggatgccacagcccagcttggccctttccttccagatcctgggtactgaaagccttagggaagctggcctgagaggggaagcggccctaagggagtgtctaagaacaaaagcgacccattcagagactgtccctgaaacctagtactgccccccatgaggaaggaacagcaatggtgtcagtatccaggctttgtacagagtgcttttctgtttagtttttactttttttgttttgtttttttaaagacgaaataaagacccaggggagaatgggtgttgtatggggaggcaagtgtggggggtccttctccacacccactttgtccatttgcaaatatattttggaaaac

SEQ ID NO: 5 - DNA, 310 bp, Artificial
  Synthetic construct
tgaaccagaaggccaagtccgcagaagccctgatgtgtcctcagggagcagggaaggcctgacttctgctggcatcaagaggtgggagggccctccgaccacttccaggggaacctgccatgccaggaacctgtcctaaggaaccttccttcctgcttgagttcccagatggctggaaggggtccagcctcgttggaagaggaacagcactggggagtctttgtggattctgaggccctgcccaatgagactctagggtccagtggatgccacagcccagcttggccctttccttccagatcctgggtac

SEQ ID NO: 6 - DNA, 219 bp, Artificial
  Synthetic construct
ggctgcttgaggaagtataagaatgaagttgtgaagctgagattcccctccattgggaccggagaaaccaggggagccccccgggcagccgcgcgccccttcccacggggccctttactgcgccgcgcgcccggcccccacccctcgcagcaccccgcgccccgcgccctcccagccgggtccagccggagccatggggccggagccgcagtgagcacc

SEQ ID NO: 7 - DNA, 104 bp, Artificial
  Synthetic construct
ccttccttcctgcttgagttcccagatggctggaaggggtccagcctcgttggaagaggaacagcactggggagtctttgtggattctgaggccctgcccaatg

SEQ ID NO: 8 - DNA, 73 bp, Artificial
  Synthetic construct
cttttctgtttagtttttactttttttgttttgtttttttaaagatgaaataaagacccaggggagaatgggt

SEQ ID NO: 9 - DNA, 73 bp, Artificial
  Synthetic construct
cttttctgtttagtttttactttttttgttttgtttttttaaagatgaaataaagacccagggggagatgggt

SEQ ID NO: 10 - DNA, 73 bp, Artificial
  Synthetic construct
cttttctgtttagtttttactttttttgttttgtttttttaaagacgaaataaagacccagggggagatgggt

SEQ ID NO: 11 - DNA, 73 bp, Artificial
  Synthetic construct
cttttctgtttagtttttactttttttgttttgtttttttaaagacgaaataaagacccagggggggatgggt

SEQ ID NO: 12 - DNA, 73 bp, Artificial
  Synthetic construct
cttttctgtttagtttttactttttttgttttgtttttttaaagacgaaataaagacccaggggaaaatgggt

SEQ ID NO: 13 - DNA, 73 bp, Artificial
  Synthetic construct
cttttctgtttagtttttactttttttgttttgtttttttaaagacgaaataaagacccaggggaagatgggt

SEQ ID NO: 14 - DNA, 73 bp, Artificial
  Synthetic construct
cttttctgtttagtttttactttttttgttttgtttttttaaagacgaaataaagacccagggggaaatgggt

SEQ ID NO: 15 - DNA, 73 bp, Artificial
  Synthetic construct
cttttctgtttagtttttactttttttgttttgtttttttaaagacgaaataaagacccaggggaggatgggt

SEQ ID NO: 16 - DNA, 73 bp, Artificial
  Synthetic construct
cttttctgtttagtttttactttttttgttttgtttttttaaagacgaaataaagacccaggggggaatgggt

SEQ ID NO: 17 - DNA, 73 bp, Artificial
  Synthetic construct
cttttctgtttagtttttactttttttgttttgtttttttaaagatgaaataaagacccagggggggatgggt

SEQ ID NO: 18 - DNA, 73 bp, Artificial
  Synthetic construct
cttttctgtttagtttttactttttttgttttgtttttttaaagatgaaataaagacccaggggaaaatgggt

SEQ ID NO: 19 - DNA, 73 bp, Artificial
  Synthetic construct
cttttctgtttagtttttactttttttgttttgtttttttaaagatgaaataaagacccaggggaagatgggt

SEQ ID NO: 20 - DNA, 73 bp, Artificial
  Synthetic construct
cttttctgtttagtttttactttttttgttttgtttttttaaagatgaaataaagacccagggggaaatgggt

SEQ ID NO: 21 - DNA, 73 bp, Artificial
  Synthetic construct
cttttctgtttagtttttactttttttgttttgtttttttaaagatgaaataaagacccaggggaggatgggt

SEQ ID NO: 22 - DNA, 73 bp, Artificial
  Synthetic construct
cttttctgtttagtttttactttttttgttttgtttttttaaagatgaaataaagacccaggggggaatgggt

SEQ ID NO: 23 - DNA, 540 bp, Artificial
  Synthetic construct
tgaaccagaaggccaagtccgcagaagccctgatgtgtcctcagggagcagggaaggcctgacttctgctggcatcaagaggtgggagggccctccgaccacttccaggggaacctgccatgccaggaacctgtcctaaggaaccttccttcctgcttgagttcccagatggctggaaggggtccagcctcgttggaagaggaacagcactggggagtctttgtggattctgaggccctgcccaatgagactctagggtccagtggatgccacagcccagcttggccctttccttccagatcctgggtactgaaagccttagggaagctggcctgagaggggaagcggccctaagggagtgtctaagaacaaaagcgacccattcagagactgtccctgaaacctagtactgccccccatgaggaaggaacagcaatggtgtcagtatccaggctttgtacagagtgcttttctgtttagtttttactttttttgttttgtttttttaaagacgaaataaagacccaggggagaatgggt

SEQ ID NO: 24 - DNA, 468 bp, Artificial
  Synthetic construct
tgaaccagaaggccaagtccgcagaagccctgatgtgtcctcagggagcagggaaggcctgacttctgctggcatcaagaggtgggagggccctccgaccacttccaggggaacctgccatgccaggaacctgtcctaaggaaccttccttcctgcttgagttcccagatggctggaaggggtccagcctcgttggaagaggaacagcactggggagtctttgtggattctgaggccctgcccaatgagactctagggtccagtggatgccacagcccagcttggccctttccttccagatcctgggtactgaaagccttagggaagctggcctgagaggggaagcggccctaagggagtgtctaagaacaaaagcgacccattcagagactgtccctgaaacctagtactgccccccatgaggaaggaacagcaatggtgtcagtatccaggctttgtacagagtgc

SEQ ID NO: 25 - DNA, 410 bp, Artificial
  Synthetic construct
tgaaccagaaggccaagtccgcagaagccctgatgtgtcctcagggagcagggaaggcctgacttctgctggcatcaagaggtgggagggccctccgaccacttccaggggaacctgccatgccaggaacctgtcctaaggaaccttccttcctgcttgagttcccagatggctggaaggggtccagcctcgttggaagaggaacagcactggggagtctttgtggattctgaggccctgcccaatgagactctagggtccagtggatgccacagcccagcttggccctttccttccagatcctgggtactgaaagccttagggaagctggcctgagaggggaagcggccctaagggagtgtctaagaacaaaagcgacccattcagagactgtccctgaaacctagtac

SEQ ID NO: 26 - DNA, 310 bp, Artificial; identical to SEQ ID NO: 5
  Synthetic construct

SEQ ID NO: 27 - DNA, 210 bp, Artificial
  Synthetic construct
tgaaccagaaggccaagtccgcagaagccctgatgtgtcctcagggagcagggaaggcctgacttctgctggcatcaagaggtgggagggccctccgaccacttccaggggaacctgccatgccaggaacctgtcctaaggaaccttccttcctgcttgagttcccagatggctggaaggggtccagcctcgttggaagaggaacagcac

SEQ ID NO: 28 - DNA, 110 bp, Artificial
  Synthetic construct
tgaaccagaaggccaagtccgcagaagccctgatgtgtcctcagggagcagggaaggcctgacttctgctggcatcaagaggtgggagggccctccgaccacttccaggg

SEQ ID NO: 29 - DNA, 502 bp, Artificial
  Synthetic Construct
cctgccatgccaggaacctgtcctaaggaaccttccttcctgcttgagttcccagatggctggaaggggtccagcctcgttggaagaggaacagcactggggagtctttgtggattctgaggccctgcccaatgagactctagggtccagtggatgccacagcccagcttggccctttccttccagatcctgggtactgaaagccttagggaagctggcctgagaggggaagcggccctaagggagtgtctaagaacaaaagcgacccattcagagactgtccctgaaacctagtactgccccccatgaggaaggaacagcaatggtgtcagtatccaggctttgtacagagtgcttttctgtttagtttttactttttttgttttgtttttttaaagacgaaataaagacccaggggagaatgggtgttgtatggggaggcaagtgtggggggtccttctccacacccactttgtccatttgcaaatatattttggaaaac

SEQ ID NO: 30 - DNA, 11 bp, Artificial
  Synthetic construct
gtttttttaaa